SEQUENCE LISTING (114 sequences)

SEQ ID NO: 1 - DNA, 22 bp, Artificial Sequence
  Synthetic primer sequence
caacagcaactcccactcttcc

SEQ ID NO: 2 - DNA, 21 bp, Artificial Sequence
  Synthetic primer sequence
atgtaggccatgaggtccacc

SEQ ID NO: 3 - DNA, 20 bp, Artificial Sequence
  Synthetic primer sequence
gccactccagctacttcttc

SEQ ID NO: 4 - DNA, 20 bp, Artificial Sequence
  Synthetic primer sequence
cacatgtaacctgttgccac

SEQ ID NO: 5 - DNA, 20 bp, Artificial Sequence
  Synthetic primer sequence
gaggggaaattgaggaaagc

SEQ ID NO: 6 - DNA, 20 bp, Artificial Sequence
  Synthetic primer sequence
ccttcttgtcctttggtgtg

SEQ ID NO: 7 - DNA, 20 bp, Artificial Sequence
  Synthetic primer sequence
actcttccaaagccagactc

SEQ ID NO: 8 - DNA, 20 bp, Artificial Sequence
  Synthetic primer sequence
gcattccaatccaggtttcc

SEQ ID NO: 9 - DNA, 20 bp, Artificial Sequence
  Synthetic primer sequence
tgatcctcaaaaaagccacc

SEQ ID NO: 10 - DNA, 20 bp, Artificial Sequence
  Synthetic primer sequence
cgaagctgttcgagtttgtg

SEQ ID NO: 11 - DNA, 20 bp, Artificial Sequence
  Synthetic primer sequence
acacacacacacacacacac

SEQ ID NO: 12 - DNA, 20 bp, Artificial Sequence
  Synthetic primer sequence
tgggatgggccttgttttgg

SEQ ID NO: 13 - DNA, 20 bp, Artificial Sequence
  Synthetic primer sequence
aggggcgatccagaacaacg

SEQ ID NO: 14 - DNA, 20 bp, Artificial Sequence
  Synthetic primer sequence
atggtcgtaggggctttctc

SEQ ID NO: 15 - DNA, 20 bp, Artificial Sequence
  Synthetic primer sequence
tcagtgggttgacacggatg

SEQ ID NO: 16 - DNA, 21 bp, Artificial Sequence
  Synthetic primer sequence
tgatcttaaaggccaccaagc

SEQ ID NO: 17 - DNA, 20 bp, Artificial Sequence
  Synthetic primer sequence
tcgctaccttcgagaagctg

SEQ ID NO: 18 - DNA, 20 bp, Artificial Sequence
  Synthetic primer sequence
attcttccccaacctccacc

SEQ ID NO: 19 - DNA, 20 bp, Artificial Sequence
  Synthetic primer sequence
aagaaagcactccctgactg

SEQ ID NO: 20 - DNA, 20 bp, Artificial Sequence
  Synthetic primer sequence
tcctcaaatgccatcccctc

SEQ ID NO: 21 - DNA, 20 bp, Artificial Sequence
  Synthetic primer sequence
gcttcgcaagaagaagaacc

SEQ ID NO: 22 - DNA, 20 bp, Artificial Sequence
  Synthetic primer sequence
agatctcctcatcaccagcc

SEQ ID NO: 23 - DNA, 19 bp, Artificial Sequence
  Synthetic primer sequence
tttggcaatgtggaaggac

SEQ ID NO: 24 - DNA, 20 bp, Artificial Sequence
  Synthetic primer sequence
tgagggaagatattctgggc

SEQ ID NO: 25 - DNA, 20 bp, Artificial Sequence
  Synthetic primer sequence
gtagcagaaacctcatcacc

SEQ ID NO: 26 - DNA, 20 bp, Artificial Sequence
  Synthetic primer sequence
aggccaacataatcagcaac

SEQ ID NO: 27 - DNA, 21 bp, Artificial Sequence
  Synthetic primer sequence
gtcagctcaatcctaaggatg

SEQ ID NO: 28 - DNA, 20 bp, Artificial Sequence
  Synthetic primer sequence
atttactgcaaacacagggg

SEQ ID NO: 29 - DNA, 20 bp, Artificial Sequence
  Synthetic primer sequence
acaacgaaaaggcccccaag

SEQ ID NO: 30 - DNA, 20 bp, Artificial Sequence
  Synthetic primer sequence
taactgttctcgccgcttcc

SEQ ID NO: 31 - DNA, 21 bp, Artificial Sequence
  Synthetic primer sequence
ccgaagaactgacaaggatac

SEQ ID NO: 32 - DNA, 20 bp, Artificial Sequence
  Synthetic primer sequence
gcagtctgcaaactttgaac

SEQ ID NO: 33 - DNA, 22 bp, Artificial Sequence
  Synthetic primer sequence
gggcaagtttttccaatatgac

SEQ ID NO: 34 - DNA, 19 bp, Artificial Sequence
  Synthetic primer sequence
cctctctgaacgcttcaag

SEQ ID NO: 35 - DNA, 20 bp, Artificial Sequence
  Synthetic primer sequence
tgttttccagtcctcctcag

SEQ ID NO: 36 - DNA, 20 bp, Artificial Sequence
  Synthetic primer sequence
taccctaaccccactccttg

SEQ ID NO: 37 - DNA, 20 bp, Artificial Sequence
  Synthetic primer sequence
tgctgccaactcagtgttcg

SEQ ID NO: 38 - DNA, 20 bp, Artificial Sequence
  Synthetic primer sequence
tctcctcgttgtgctcatcc

SEQ ID NO: 39 - DNA, 21 bp, Artificial Sequence
  Synthetic primer sequence
gcagttcaaatacacacagcc

SEQ ID NO: 40 - DNA, 21 bp, Artificial Sequence
  Synthetic primer sequence
tgagtctcctgaatacgaacc

SEQ ID NO: 41 - DNA, 20 bp, Artificial Sequence
  Synthetic primer sequence
aatttcctctgctgacaacc

SEQ ID NO: 42 - DNA, 20 bp, Artificial Sequence
  Synthetic primer sequence
gcatcccagattttcactcc

SEQ ID NO: 43 - DNA, 20 bp, Artificial Sequence
  Synthetic primer sequence
tgcagcacttacacagtgtc

SEQ ID NO: 44 - DNA, 20 bp, Artificial Sequence
  Synthetic primer sequence
tttgtcggagtagtccaccc

SEQ ID NO: 45 - DNA, 21 bp, Artificial Sequence
  Synthetic primer sequence
tctttccctccttctcaccac

SEQ ID NO: 46 - DNA, 20 bp, Artificial Sequence
  Synthetic primer sequence
aggaccaacttagtccccac

SEQ ID NO: 47 - DNA, 20 bp, Artificial Sequence
  Synthetic primer sequence
agggatgtaaaccaggatgg

SEQ ID NO: 48 - DNA, 20 bp, Artificial Sequence
  Synthetic primer sequence
agaaactcccaggtgttagg

SEQ ID NO: 49 - DNA, 20 bp, Artificial Sequence
  Synthetic primer sequence
gacctgccgtctagaaaaac

SEQ ID NO: 50 - DNA, 20 bp, Artificial Sequence
  Synthetic primer sequence
ttgaagtcagaggagaccac

SEQ ID NO: 51 - DNA, 21 bp, Artificial Sequence
  Synthetic primer sequence
gtatcagcatgatggtagcag

SEQ ID NO: 52 - DNA, 20 bp, Artificial Sequence
  Synthetic primer sequence
agcaacactaaaatcagccc

SEQ ID NO: 53 - DNA, 21 bp, Artificial Sequence
  Synthetic primer sequence
tgagtccttctgagatgatgg

SEQ ID NO: 54 - DNA, 20 bp, Artificial Sequence
  Synthetic primer sequence
tgagaaccgagttcaaggtg

SEQ ID NO: 55 - DNA, 21 bp, Artificial Sequence
  Synthetic primer sequence
ctgaaagcatcttaacccctc

SEQ ID NO: 56 - DNA, 21 bp, Artificial Sequence
  Synthetic primer sequence
catcttagtgtgtggtcttcc

SEQ ID NO: 57 - DNA, 20 bp, Artificial Sequence
  Synthetic primer sequence
gccacgtctccacacatcag

SEQ ID NO: 58 - DNA, 22 bp, Artificial Sequence
  Synthetic primer sequence
tcttggcagcaggatagtcctt

SEQ ID NO: 59 - DNA, 21 bp, Artificial Sequence
  Synthetic primer sequence
cagaagaacagacaaggacac

SEQ ID NO: 60 - DNA, 20 bp, Artificial Sequence
  Synthetic primer sequence
tgcagtccgcaaacttttac

SEQ ID NO: 61 - DNA, 19 bp, Artificial Sequence
  Synthetic primer sequence
tggagacgtactgtgctac

SEQ ID NO: 62 - DNA, 21 bp, Artificial Sequence
  Synthetic primer sequence
ccaggtgtcatattggaagaa

SEQ ID NO: 63 - DNA, 20 bp, Artificial Sequence
  Synthetic primer sequence
gcttgtccaagacctgaagg

SEQ ID NO: 64 - DNA, 21 bp, Artificial Sequence
  Synthetic primer sequence
acagtagaatagatgtgcccc

SEQ ID NO: 65 - DNA, 20 bp, Artificial Sequence
  Synthetic primer sequence
aagaaagcactccctgactg

SEQ ID NO: 66 - DNA, 20 bp, Artificial Sequence
  Synthetic primer sequence
tcctcaaatgccatcccctc

SEQ ID NO: 67 - DNA, 20 bp, Artificial Sequence
  Synthetic primer sequence
aagtttgccatgtacccacc

SEQ ID NO: 68 - DNA, 20 bp, Artificial Sequence
  Synthetic primer sequence
ttagccagcagctcagtcag

SEQ ID NO: 69 - DNA, 20 bp, Artificial Sequence
  Synthetic primer sequence
aagtctctgctgctcaagcc

SEQ ID NO: 70 - DNA, 20 bp, Artificial Sequence
  Synthetic primer sequence
acgaacacgaagtcgttgtc

SEQ ID NO: 71 - DNA, 20 bp, Artificial Sequence
  Synthetic primer sequence
gaattccctctgctgacaac

SEQ ID NO: 72 - DNA, 20 bp, Artificial Sequence
  Synthetic primer sequence
gcatcccagattttcactcc

SEQ ID NO: 73 - DNA, 21 bp, Artificial Sequence
  Synthetic primer sequence
tcgataaagaggaggaagctg

SEQ ID NO: 74 - DNA, 20 bp, Artificial Sequence
  Synthetic primer sequence
catactgagtgtcaccgttg

SEQ ID NO: 75 - DNA, 21 bp, Artificial Sequence
  Synthetic primer sequence
cctctcgtaaacggaaagaag

SEQ ID NO: 76 - DNA, 20 bp, Artificial Sequence
  Synthetic primer sequence
tgctatgtcagattgtcccc

SEQ ID NO: 77 - DNA, 19 bp, Artificial Sequence
  Synthetic primer sequence
tgctgccaactcagtgttc

SEQ ID NO: 78 - DNA, 20 bp, Artificial Sequence
  Synthetic primer sequence
tctcctcatthtgctcatcc

SEQ ID NO: 79 - DNA, 21 bp, Artificial Sequence
  Synthetic primer sequence
accagacacaagattcgagag

SEQ ID NO: 80 - DNA, 20 bp, Artificial Sequence
  Synthetic primer sequence
tccaagcacagaaaagttcc

SEQ ID NO: 81 - DNA, 20 bp, Artificial Sequence
  Synthetic primer sequence
tgtccaacctgcgctacaac

SEQ ID NO: 82 - DNA, 20 bp, Artificial Sequence
  Synthetic primer sequence
tgctgaaggtggtcatgtcg

SEQ ID NO: 83 - DNA, 21 bp, Artificial Sequence
  Synthetic primer sequence
ccctgcaagccctaataaatg

SEQ ID NO: 84 - DNA, 21 bp, Artificial Sequence
  Synthetic primer sequence
caattaatcaccaccaccacc

SEQ ID NO: 85 - DNA, 20 bp, Artificial Sequence
  Synthetic primer sequence
tctccaacatccaccccttc

SEQ ID NO: 86 - DNA, 20 bp, Artificial Sequence
  Synthetic primer sequence
tcgccaaaccagggataaag

SEQ ID NO: 87 - DNA, 20 bp, Artificial Sequence
  Synthetic primer sequence
gtaggaaaggtgagaggcag

SEQ ID NO: 88 - DNA, 22 bp, Artificial Sequence
  Synthetic primer sequence
ccccaattcaagagtgacaaag

SEQ ID NO: 89 - DNA, 20 bp, Artificial Sequence
  Synthetic primer sequence
acccaaagccctcttctgac

SEQ ID NO: 90 - DNA, 22 bp, Artificial Sequence
  Synthetic primer sequence
tggcagcttactttccaatacc

SEQ ID NO: 91 - DNA, 20 bp, Artificial Sequence
  Synthetic primer sequence
acccaacagcaacaacaaac

SEQ ID NO: 92 - DNA, 20 bp, Artificial Sequence
  Synthetic primer sequence
aatggggggcatcttgatcc

SEQ ID NO: 93 - DNA, 20 bp, Artificial Sequence
  Synthetic primer sequence
ttccccagccttagagagac

SEQ ID NO: 94 - DNA, 20 bp, Artificial Sequence
  Synthetic primer sequence
gttcttgccctgcgtatatc

SEQ ID NO: 95 - DNA, 24 bp, Artificial Sequence
  Synthetic primer sequence
aggggtagtataggtaggaggatt

SEQ ID NO: 96 - DNA, 23 bp, Artificial Sequence
  Synthetic primer sequence
aaatctcaaaaaccaaaccaaac

SEQ ID NO: 97 - DNA, 20 bp, Artificial Sequence
  Synthetic primer sequence
gttgctgggaattgaactcg

SEQ ID NO: 98 - DNA, 20 bp, Artificial Sequence
  Synthetic primer sequence
ttcacacacaccccatctcc

SEQ ID NO: 99 - DNA, 1812 bp, Homo sapiens
atgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaa

SEQ ID NO: 100 - DNA, 3342 bp, Homo sapiens
atgtggcaggagacgtttttcagaatggagaatctgcaactcatcataatcctgattcttggatctcccagtcagcttcatttccccgtaatcagaaacaaccaggggtggactctttatcaccagtggcctcacttcctaaacagattttccagccgtctgtccaacagcagcctactaaaccagttaaagttcacttgtgcaaactgcaaaaaacctttacagaagggccagacagcttatcaacgaaaaggatcagctcacctcttttgttctaccacctgcctttcttccttctcccacaagcctgctccaaagaaactctgtgttatgtgtaaaaaagatataactacaatgaaaggaaccattgttgctcaagtggattcaagtgagtccttccaggaattctgtagtacatcttgtttatctctctatgaagacaaacagaatcctactaaaggagctctaaataaatcaagatgtacaatctgtggtaaactaactgagattcgccatgaagtcagctttaaaaatatgactcataagctgtgcagtgaccactgctttaatagatatagaatggccaatggtttaataatgaattgctgtgaacagtgtggagagtacttgcccagtaaaggtgctggaaataatgttctggtgattgatggtcaacagaaaagattttgctgtcaaagttgtgtcagtgaatacaaacaggtaggtagccatccaagcttcctgaaggaggttcgagatcacatgcaggactcttttttgatgcagcctgagaaatatggaaaactgacaacttgtactggttgccgaacacagtgcaggttttttgatatgactcagtgtataggtcctaatggatatatggagccatattgttcaactgcttgtatgaacagtcacaagacaaaatatgcaaaatcacaaagtttgggaattatttgccatttttgtaagcgaaactctttacctcaataccaagccacaatgcctgatggaaaactgtacaacttttgcaattccagttgtgtggctaaatttcaggctctaagtatgcagtcatctccaaatggccagtttgtagcgccaagtgatattcagttgaaatgcaactactgcaaaaattccttttgttcaaaaccagaaatcctggaatgggagaacaaagtgcatcagttctgcagcaaaacttgttcagatgactataagaagttgcattgcatagttacatattgcgaatactgtcaagaggagaagactcttcatgaaacagtaaatttctctggcgttaagagacctttctgtagtgaaggctgcaaattattatacaaacaggattttgccagacgtttaggattgagatgtgttacttgcaactattgttctcagctatgtaagaagggagcaactaaagaactcgatggtgttgtgagagatttctgcagtgaagattgctgtaaaaaatttcaggattggtactacaaggctgcaaggtgtgactgttgtaaatctcaaggaactcttaaagagcgagttcagtggcgtggggaaatgaaacatttctgtgatcaacattgcttactgcgtttctactgtcaacaaaatgagcccaacatgacaactcagaaaggacctgaaaacttacattatgatcagggttgtcagacatctcgaaccaaaatgacaggttcagcaccacccccttctccaacacctaacaaagagatgaagaacaaagcagttctttgcaaacctttaacaatgacaaaagctacttactgtaaacctcacatgcagaccaaatcttgtcagacagatgatacttggaggacagaatatgttccagtgcctatccctgtgcctgtgtatatcccagttcctatgcacatgtacagtcagaatattcctgttcctactacagttcctgttcctgtgccagttcctgtttttctgcctgctccattggacagcagtgagaagattcctgcagcaattgaggagctaaaaagcaaggtttcttcagatgctcttgatacagagttgcttacaatgacggatatgatgagtgaagacgaggggaaaacagagacaaccaacatcaacagtgtaattattgaaacagatataattggttcagaccttttgaagaactctgacccagagacacagtccagcatgcctgatgtaccatatgaaccagatttggatatcgaaatagattttcccagagctgctgaggagcttgatatggaaaatgaatttttattaccacctgtttttggcgaagaatatgaggaacagcccagacctcgatctaaaaaaaagggagccaagagaaaggctgtatcaggataccagtctcatgatgatagttctgacaattcagaatgcagctttcctttcaaatatacgtatggcgtaaatgcatggaaacactgggtcaaaactaggcaacttgatgaagatcttctggtattagatgagttaaaatcttctaaatcagtaaagttaaaagaggatctactctctcacaccacagctgagcttaactatgggttagctcattttgtcaatgagatccgacggccaaatggagagaattatgcacctgacagcatctattacctttgccttggaatacaggagtatttgtgtggaagtaatcgaaaagacaacatatttattgatcctggataccaaacatttgagcaagaattgaataaaatactgcgaagctggcaaccaagcatacttccagatgggtcaatattctctcgagttgaagaagactatctctggaggataaaacaactaggatcacactctccagtagctcttctgaatacactgttctactttaacactaagtattttggcctgaaaacagtggaacaacacttaagactttcctttggcactgtgtttaggcattggaaaaaaaatcctttaacgatggaaaacaaagcgtgtcttcgataccaagtgtcttccttgtgtggaacagataatgaagataaaattactactggaaaaagaaaacatgaagatgatgagccagtatttgaacaaattgaaaacacagccaatccttccagatgtcctgtgaaaatgtttgaatgctacttgtctaaaagtccacagaatcttaatcagaggatggatgttttttatttgcaaccagaatgctctagttctacagatagccctgtctggtatacgtctacttcactggaccgaaacaccttggaaaatatgcttgtacgggttcttctagtaaaagatatttatgataaagacaattatgaactggatgaagacacagactaa

SEQ ID NO: 101 - DNA, 4134 bp, Homo sapiens
atggacacaagttcagtgggaggattagaattgactgatcagactcctgttttattagggagtacggccatggcaactagtctcacgaatgtaggaaactcatttagtggtccagctaatcctttagtgtctagatctaataagtttcagaactcgtcagtggaagatgatgatgatgttgtttttatcgaacctgtacaacctcccccaccttctgtaccagtggtagctgatcaaagaaccataacatttacatcatcaaaaaatgaagaactacaaggaaatgattccaaaattactccttcctcaaaagagttggcatctcagaagggaagtgtaagtgagacaattgtcattgatgatgaagaggacatggaaacaaatcaagggcaagagaaaaattcctccaattttattgaacgaagacctcctgagactaaaaacagaaccaatgatgtggatttctccacttccagtttttcaagaagtaaggtaaatgcaggaatgggtaatagtggtatcaccacagaaccagactctgaaattcagattgctaatgttacaactttagaaacaggtgtaagctctgtgaatgatggccaattagaaaatactgacgggcgagatatgaacttaatgattacacatgtaacatcactgcagaataccaacttgggagatgtctctaacggactgcagtcaagtaattttggtgttaatatacaaacatacaccccatctttaacttcacagaccaagactggagtaggaccttttaatcctggtagaatgaatgtggcaggagacgtttttcagaatggagaatctgcaactcatcataatcctgattcttggatctcccagtcagcttcatttccccgtaatcagaaacaaccaggggtggactctttatcaccagtggcctcacttcctaaacagattttccagccgtctgtccaacagcagcctactaaaccagttaaagtcacttgtgcaaactgcaaaaaacctttacagaagggccagacagcttatcaacgaaaaggatcagctcacctcttttgttctaccacctgcctttcttccttctcccacaagcctgctccaaagaaactctgtgttatgtgtaaaaaagatataactacaatgaaaggaaccattgttgctcaagtggattcaagtgagtccttccaggaattctgtagtacatcttgtttatctctctatgaagacaaacagaatcctactaaaggagctctaaataaatcaagatgtacaatctgtggtaaactaactgagattcgccatgaagtcagctttaaaaatatgactcataagctgtgcagtgaccactgctttaatagatatagaatggccaatggtttaataatgaattgctgtgaacagtgtggagagtacttgcccagtaaaggtgctggaaataatgttctggtgattgatggtcaacagaaaagattttgctgtcaaagttgtgtcagtgaatacaaacaggtaggtagccatccaagcttcctgaaggaggttcgagatcacatgcaggactctttcttaatgcagcctgagaaatatggaaaactgacaacttgtactggttgccgaacacagtgcaggttttttgatatgactcagtgtataggtcctaatggatatatggagccatattgttcaactgcttgtatgaacagtcacaagacaaaatatgcaaaatcacaaagtttgggaattatttgccatttttgtaagcgaaactctttacctcaataccaagccacaatgcctgatggaaaactgtacaacttttgcaattccagttgtgtggctaaatttcaggctctaagtatgcagtcatctccaaatggccagtttgtagcgccaagtgatattcagttgaaatgcaactactgcaaaaattccttttgttcaaaaccagaaatcctggaatgggagaacaaagtgcatcagttctgcagcaaaacttgttcagatgactataagaagttgcattgcatagttacatattgcgaatactgtcaagaggagaagactcttcatgaaacagtaaatttctctggcgttaagagacctttctgtagtgaaggctgcaaattattatacaaacaggattttgccagacgtttaggattgagatgtgttacttgcaactattgttctcagctatgtaagaagggagcaactaaagaactcgatggtgttgtgagagatttctgcagtgaagattgctgtaaaaaatttcaggattggtactacaaggctgcaaggtgtgactgttgtaaatctcaaggaactcttaaagagcgagttcagtggcgtggggaaatgaaacatttctgtgatcaacattgcttactgcgtttctactgtcaacaaaatgagcccaacatgacaactcagaaaggacctgaaaacttacattatgatcagggttgtcagacatctcgaaccaaaatgacaggttcagcaccacccccttctccaacacctaacaaagagatgaagaacaaagcagttctttgcaaacctttaacaatgacaaaagctacttactgtaaacctcacatgcagaccaaatcttgtcagacagatgatacttggaggacagaatatgttccagtgcctatccctgtgcctgtgtatatcccagttcctatgcacatgtacagtcagaatattcctgttcctactacagttcctgttcctgtgccagttcctgtttttctgcctgctccattggacagcagtgagaagattcctgcagcaattgaggagctaaaaagcaaggtttcttcagatgctcttgatacagagttgcttacaatgacggatatgatgagtgaagacgaggggaaaacagagacaaccaacatcaacagtgtaattattgaaacagatataattggttcagaccttttgaagaactctgacccagagacacagtccagcatgcctgatgtaccatatgaaccagatttggatatcgaaatagattttcccagagctgctgaggagcttgatatggaaaatgaatttttattaccacctgtttttggcgaagaatatgaggaacagcccagacctcgatctaaaaaaaagggagccaagagaaaggctgtatcaggataccagtctcatgatgatagttctgacaattcagaatgcagctttcctttcaaatatacgtatggcgtaaatgcatggaaacactgggtcaaaactaggcaacttgatgaagatcttctggtattagatgagttaaaatcttctaaatcagtaaagttaaaagaggatctactctctcacaccacagctgagcttaactatgggttagctcattttgtcaatgagatccgacggccaaatggagagaattatgcacctgacagcatctattacctttgccttggaatacaggagtatttgtgtggaagtaatcgaaaagacaacatatttattgatcctggataccaaacatttgagcaagaattgaataaaatactgcgaagctggcaaccaagcatacttccagatgggtcaatattctctcgagttgaagaagactatctctggaggataaaacaactaggatcacactctccagtagctcttctgaatacactgttctactttaacactaagtattttggcctgaaaacagtggaacaacacttaagactttcctttggcactgtgtttaggcattggaaaaaaaatcctttaacgatggaaaacaaagcgtgtcttcgataccaagtgtcttccttgtgtggaacagataatgaagataaaattactactggaaaaagaaaacatgaagatgatgagccagtatttgaacaaattgaaaacacagccaatccttccagatgtcctgtgaaaatgtttgaatgctacttgtctaaaagtccacagaatcttaatcagaggatggatgttttttatttgcaaccagaatgctctagttctacagatagccctgtctggtatacgtctacttcactggaccgaaacaccttggaaaatatgcttgtacgggttcttctagtaaaagatatttatgataaagacaattatgaactggatgaagacacagactaa

SEQ ID NO: 102 - DNA, 2220 bp, Homo sapiens
atggcgcctcagaagcacggcggtgggggagggggcggctcggggcccagcgcggggtccgggggaggcggcttcgggggttcggcggcggtggcggcggcgacggcttcgggcggcaaatccggcggcgggagctgtggagggggtggcagttactcggcctcctcctcctcctccgcggcggcagcggcgggggctgcggtgttaccggtgaagaagccgaaaatggagcacgtccaggctgaccacgagcttttcctccaggcctttgagaagccaacacagatctatagatttcttcgaactcggaatctcatagcaccaatatttttgcacagaactcttacttacatgtctcatcgaaactccagaacaaacatcaaaaggaaaacatttaaagttgatgatatgttatcaaaagtagagaaaatgaaaggagagcaagaatctcatagcttgtcagctcatttgcagcttacgtttactggtttcttccacaaaaatgataagccatcaccaaactcagaaaatgaacaaaattctgttaccctggaagtcctgcttgtgaaagtttgccacaaaaaaagaaaggatgtaagttgtccaataaggcaagttcccacaggtaaaaagcaggtgcctttgaatcctgacctcaatcaaacaaaacccggaaatttcccgtcccttgcagtttccagtaatgaatttgaacctagtaacagccatatggtgaagtcttactcgttgctatttagagtgactcgtccaggaagaagagagtttaatggaatgattaatggagaaaccaatgaaaatattgatgtcaatgaagagcttccagccagaagaaaacgaaatcgtgaggatggggaaaagacatttgttgcacaaatgacagtatttgataaaaacaggcgcttacagcttttagatggggaatatgaagtagccatgcaggaaatggaagaatgtccaataagcaagaaaagagcaacatgggagactattcttgatgggaagaggctgcctccattcgaaacattttctcagggacctacgttgcagttcactcttcgttggacaggagagaccaatgataaatctacggctcctattgccaaacctcttgccactagaaattcagagagtctccatcaggaaaacaagcctggttcagttaaacctactcaaactattgctgttaaagaatcattgactacagatctacaaacaagaaaagaaaaggatactccaaatgaaaaccgacaaaaattaagaatattttatcagtttctctataacaacaatacaaggcaacaaactgaagcaagagatgacctgcattgcccttggtgtactctgaactgccgcaaactttatagtttactcaagcatcttaaactctgccatagcagatttatcttcaactatgtttatcatccaaaaggtgctaggatagatgtttctatcaatgagtgttatgatggctcctatgcaggaaatcctcaggatattcatcgccaacctggatttgcttttagtcgcaacggaccagttaagagaacacctatcacacatattcttgtgtgcaggccaaaacgaacaaaagcaagcatgtctgaatttcttgaatctgaagatggggaagtagaacagcaaagaacatatagtagtggccacaatcgtctgtatttccatagtgatacctgcttacctctccgtccacaagaaatggaagtagatagtgaagatgaaaaggatcctgaatggctaagagaaaaaaccattacacaaattgaagagttttctgatgttaatgaaggagagaaagaagtgatgaaactctggaatctccatgtcatgaagcatgggtttattgctgacaatcaaatgaatcatgcctgtatgctgtttgtagaaaattatggacagaaaataattaagaagaatttatgtcgaaacttcatgcttcatctagtcagcatgcatgactttaatcttattagcataatgtcaatagataaagctgttaccaagctccgtgaaatgcagcaaaaattagaaaagggggaatctgcttcccctgcaaacgaagaaataactgaagaacaaaatgggacagcaaatggatttagtgaaattaactcaaaagagaaagctttggaaacagatagtgtctcaggggtttcaaaacagagcaaaaaacaaaaactctga

SEQ ID NO: 103 - DNA, 783 bp, Homo sapiens
atggatcgccactccagctacatcttcatctggctgcagctggagctctgcgccatggccgtgctgctcaccaaaggtgaaattcgatgctactgtgatgctgcccactgtgtagccactggttatatgtgtaaatctgagctcagcgcctgcttctctagacttcttgatcctcagaactcaaattccccactcacccatggctgcctggactctcttgcaagcacgacagacatctgccaagccaaacaggcccgaaaccactctggcaccaccatacccacattggaatgctgtcatgaagacatgtgcaattacagagggctgcacgatgttctctctcctcccaggggtgaggcctcaggacaaggaaacaggtatcagcatgatggtagcagaaaccttatcaccaaggtgcaggagctgacttcttccaaagagttgtggttccgggcagcggtcattgccgtgcccattgctggagggctgattttagtgttgcttattatgttggccctgaggatgcttcgaagtgaaaataagaggctgcaggatcagcggcaacagatgctctcccgtttgcactacagctttcacggacaccattccaaaaaggggcaggttgcaaagttagacttggaatgcatggtgccggtcagtgggcacgagaactgctgtctgacctgtgataaaatgagacaagcagacctcagcaacgataagatcctctcgcttgttcactggggcatgtacagtgggcacgggaagctggaattcgtatga

SEQ ID NO: 104 - DNA, 870 bp, Homo sapiens
atggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtga

SEQ ID NO: 105 - DNA, 780 bp, Homo sapiens
atggccgcgttgatgcggagcaaggattcgtcctgctgcctgctcctactggccgcggtgctgatggtggagagctcacagatcggcagttcgcgggccaaactcaactccatcaagtcctctctgggcggggagacgcctggtcaggccgccaatcgatctgcgggcatgtaccaaggactggcattcggcggcagtaagaagggcaaaaacctggggcaggcctacccttgtagcagtgataaggagtgtgaagttgggaggtattgccacagtccccaccaaggatcatcggcctgcatggtgtgtcggagaaaaaagaagcgctgccaccgagatggcatgtgctgccccagtacccgctgcaataatggcatctgtatcccagttactgaaagcatcttaacccctcacatcccggctctggatggtactcggcacagagatcgaaaccacggtcattactcaaaccatgacttgggatggcagaatctaggaagaccacacactaagatgtcacatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 106 - DNA, 1152 bp, Homo sapiens
atgaccgcgaccgaagccctcctgcgcgtcctcttgctcctgctggctttcggccacagcacctatggggctgaatgcttcccggcctgcaacccccaaaatggattctgcgaggatgacaatgtttgcaggtgccagcctggctggcagggtcccctttgtgaccagtgcgtgacctctcccggctgccttcacggactctgtggagaacccgggcagtgcatttgcaccgacggctgggacggggagctctgtgatagagatgttcgggcctgctcctcggccccctgtgccaacaacgggacctgcgtgagcctggacgatggcctctatgaatgctcctgtgcccccgggtactcgggaaaggactgccagaaaaaggacgggccctgtgtgatcaacggctccccctgccagcacggaggcacctgcgtggatgatgagggccgggcctcccatgcctcctgcctgtgcccccctggcttctcaggcaatttctgcgagatcgtggccaacagctgcacccccaacccatgcgagaacgacggcgtctgcactgacattgggggcgacttccgctgccggtgcccagccggcttcatcgacaagacctgcagccgcccggtgaccaactgcgccagcagcccgtgccagaacgggggcacctgcctgcagcacacccaggtgagctacgagtgtctgtgcaagcccgagttcacaggtctcacctgtgtcaagaagcgcgcgctgagcccccagcaggtcacccgtctgcccagcggctatgggctggcctaccgcctgacccctggggtgcacgagctgccggtgcagcagccggagcaccgcatcctgaaggtgtccatgaaagagctcaacaagaaaacccctctcctcaccgagggccaggccatctgcttcaccatcctgggcgtgctcaccagcctggtggtgctgggcactgtgggtatcgtcttcctcaacaagtgcgagacctgggtgtccaacctgcgctacaaccacatgctgcggaagaagaagaacctgctgcttcagtacaacagcggggaggacctggccgtcaacatcatcttccccgagaagatcgacatgaccaccttcagcaaggaggccggcgacgaggagatctaa

SEQ ID NO: 107 - DNA, 945 bp, Homo sapiens
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataa

SEQ ID NO: 108 - DNA, 543 bp, Homo sapiens
atgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtga

SEQ ID NO: 109 - DNA, 1398 bp, Homo sapiens
atggagaccggctttcccgcaatcatgtaccctggatcttttattgggggctggggagaagagtatctcagctgggaaggaccggggctcccagatttcgtcttccagcagcagcccgtggagtctgaagcaatgcactgcagcaaccccaagagtggagttgtgctggctacagtggcccgaggtcccgatgcttgtcagatactcaccagagccccgctgggccaggatcccccgcagaggacagtgctagggctgctaactgcaaatgggcagtacaggaggacctgtggccaggggatcacaagaatcaggtgttattctggatcagaaaatgccttccctccagctggaaagaaagcactccctgactgtggggtccaagagccccccaagcaagggtttgacatctacatggatgaactagagcagggggacagagacagctgctcggtcagagaggggatggcatttgaggatgtgtatgaagtagacaccggcacactcaagtcagacctgcacttcctgctggatttcaacacagtttcccctatgctggtagattcatctctcctctcccagtctgaagatatatccagtcttggcacagatgtgataaatgtgactgaatatgctgaagaaatttatcagtaccttagggaagctgaaataaggcacagacccaaagcacactacatgaagaagcagccagacatcacggaaggcatgcgcacgattctggtggactggctggtggaggttggggaagaatataaacttcgagcagagaccctgtatctggctgtcaacttcctggacaggttcctttcatgtatgtctgttctgagagggaaactgcagctcgtaggaacagcagctatgcttttggcttcgaaatatgaagagatatatcctcctgaagtagacgagtttgtctatatcaccgatgatacatacacaaaacgacaactgttaaaaatggaacacttgcttctgaaagttctagcttttgatctgacagtaccaaccaccaaccagtttctccttcagtacttgaggcgacaaggagtgtgcgtcaggactgagaacctggctaagtacgtagcagagctgagtctacttgaagcagatccattcttgaaatatcttccttcactgatagctgcagcagctttttgcctggcaaactatactgtgaacaagcacttttggccagaaacccttgctgcatttacagggtattcattaagtgaaattgtgccttgcctgagtgagcttcataaagcgtaccttgatataccccatcgacctcagcaagcaattagggagaagtacaaggcttcaaagtacctgtgtgtgtccctcatggagccacctgcagttcttcttctacaataa

SEQ ID NO: 110 - DNA, 2592 bp, Homo sapiens
atgtcgtccccggcgtcgaccccgagccgccgcggcagccggcgtggaagggccacccccgcccagacgcctcggagtgaggatgccaggtcatctccctctcagagacgtagaggcgaggattccacctccacgggggagttgcagccgatgccaacctcgcctggagtggacctgcagagccctgctgcgcaggacgtgctgttttccagccctccccaaatgcattcttcagctatccctcttgactttgatgttagttcaccactgacatacggcactcccagctctcgggtagagggaaccccaagaagtggtgttaggggcacacctgtgagacagaggcctgacctgggctctgcacagaagggcctgcaagtggatctgcagtctgacggggcagcagcagaagatatagtggcaagtgagcagtctctaggccaaaaacttgtgatctggggaacagatgtaaatgtggcagcatgcaaagaaaactttcagagatttcttcagcgttttattgaccctctggctaaagaagaagaaaatgttggcatagatattactgaacctctatacatgcaacgacttggggagattaatgttattggtgagccatttttaaatgtgaactgtgaacacatcaaatcatttgacaaaaatttgtacagacaactcatctcttacccacaggaagttattccaacttttgacatggctgtcaatgaaatcttctttgaccgttaccctgactcaatcttagaacatcagattcaagtaagaccattcaacgcattgaagactaagaatatgagaaacctgaatccagaagacattgaccagctcatcaccatcagcggcatggtgatcaggacatcccagctgattcccgagatgcaggaggccttcttccagtgccaagtgtgtgcccacacgacccgggtggagatggaccgcggccgcattgcagagcccagtgtgtgcgggcgctgccacaccacccacagcatggcactcatccacaaccgctccctcttctctgacaagcagatgatcaagcttcaggagtctccggaagacatgcctgcagggcagacaccacacacagttatcctgtttgctcacaatgatctcgttgacaaggtccagcctggggacagagtgaatgttacaggcatctatcgagctgtgcctattcgagtcaatccaagagtgagtaatgtgaagtctgtctacaaaacccacattgatgtcattcattatcggaaaacggatgcaaaacgtctgcatggccttgatgaagaagcagaacagaaacttttttcagagaaacgtgtggaattgcttaaggaactttccaggaaaccagacatttatgagaggcttgcttcagccttggctccaagcatttatgaacatgaagatataaagaagggaattttgcttcagctctttggcgggacaaggaaggattttagtcacactggaaggggcaaatttcgggctgagatcaacatcttgctgtgtggcgaccctggtaccagcaagtcccagctgctgcagtacgtgtacaacctcgtccccaggggccagtacacgtctgggaagggctccagtgcagttggcctcactgcgtacgtaatgaaagaccctgagacaaggcagctggtcctgcagacaggtgctcttgtcctgagtgacaacggcatctgctgtatcgatgagttcgacaagatgaatgaaagtacaagatcggtattgcatgaagtcatggaacagcagactctgtccattgcaaaggctgggatcatctgtcagctcaatgcgcgcacctctgtcctggcagcagcaaatcccattgagtctcagtggaatcctaaaaaaacaaccattgaaaacatccagctgcctcatactttattatcaaggtttgatttgatcttcctcttgctggaccctcaggacgaagcctatgacaggcgtctggctcaccacctggtcgcactgtactaccagagcgaggagcaggcagaggaggagctcctggacatggcggtgctaaaggactacattgcctacgcgcacagcaccatcatgccgcggctaagtgaggaagccagccaggctctcatcgaggcttatgtagacatgaggaagattggcagtagccggggaatggtttctgcataccctcgacagctagagtcattaatccgcttagcagaagcccatgctaaagtaagattgtctaacaaagttgaagccattgatgtggaagaggccaaacgcctccatcgggaagctctgaagcagtctgcaactgatccccggactggcatcgtggacatatctattcttactacggggatgagtgccacctctcgtaaacggaaagaagaattagctgaagcattgaaaaagcttattttatctaagggcaaaacaccagctctaaaataccagcaactttttgaagatattcggggacaatctgacatagcaattactaaagatatgtttgaagaagcactgcgtgccctggcagatgatgatttcctgacagtgactgggaagaccgtgcgcttgctctga

SEQ ID NO: 111 - DNA, 2205 bp, Homo sapiens
atgtcgggattcgacgatcctggcattttctacagcgacagcttcgggggcgacgcccaggccgacgaggggcaggcccgcaaatcgcagctgcagaggcgcttcaaggagttcctgcggcagtaccgagtgggcaccgaccgcacgggcttcaccttcaaatacagggatgaactcaagcggcattacaacctgggggagtactggattgaggtggagatggaggatctggccagctttgatgaggacctggccgactacttgtacaagcagccagccgagcacctgcagctgctggaggaagctgccaaggaggtagctgatgaggtgacccggccccggccttctggggaggaggtgctccaggacatccaggtcatgctcaagtcggacgccagcccttccagcattcgtagcctgaagtcggacatgatgtcacacctggtgaagatccctggcatcatcatcgcggcctctgcggtccgtgccaaggccacccgcatctctatccagtgccgcagctgccgcaacaccctcaccaacattgccatgcgccctggcctcgagggctatgccctgcccaggaagtgcaacacagatcaggctgggcgccccaaatgcccattggacccgtacttcatcatgcccgacaaatgcaaatgcgtggacttccagaccctgaagctgcaggagctgcctgatgcagtcccccacggggagatgcccagacacatgcagctctactgcgacaggtacctgtgtgacaaggtcgtccctgggaacagggttaccatcatgggcatctactccatcaagaagtttggcctgactaccagcaggggccgtgacagggtgggcgtgggcatccgaagctcctacatccgtgtcctgggcatccaggtggacacagatggctctggccgcagctttgctggggccgtgagcccccaggaggaggaggagttccgtcgcctggctgccctcccaaatgtctatgaggtcatctccaagagcatcgccccctccatctttgggggcacagacatgaagaaggccattgcctgcctgctctttgggggctcccgaaagaggctccctgatggacttactcgccgaggagacatcaacctgctgatgctaggggaccctgggacagccaagtcccagcttctgaagtttgtggagaagtgttctcccattggggtatacacgtctgggaaaggcagcagcgcagctggactgacagcctcggtgatgagggacccttcgtcccggaatttcatcatggagggcggagccatggtcctggccgatggtggggtcgtctgtattgacgagtttgacaagatgcgagaagatgaccgtgtggcaatccacgaagccatggagcagcagaccatctctatcgccaaggctgggatcaccaccaccctgaactcccgctgctccgtcctggctgctgccaactcagtgttcggccgctgggatgagacgaagggggaggacaacattgacttcatgcccaccatcttgtcgcgcttcgacatgatcttcatcgtcaaggatgagcacaatgaggagagggatgtgatgctggccaagcatgtcatcactctgcacgtgagcgcactgacacagacacaggctgtggagggcgagattgacctggccaagctgaagaagtttattgcctactgccgagtgaagtgtggcccccggctgtcagcagaggctgcagagaaactgaagaaccgctacatcatcatgcggagcggggcccgtcagcacgagagggacagtgaccgccgctccagcatccccatcactgtgcggcagctggaggccattgtgcgcatcgcggaagccctcagcaagatgaagctgcagcccttcgccacagaggcagatgtggaggaggccctgcggctcttccaagtgtccacgttggatgctgccttgtccggtaccctgtcaggggtggagggcttcaccagccaggaggaccaggagatgctgagccgcatcgagaagcagctcaagcgccgctttgccattggctcccaggtgtctgagcacagcatcatcaaggacttcaccaagcagaaatacccggagcacgccatccacaaggtgctgcagctcatgctgcggcgcggcgagatccagcatcgcatgcagcgcaaggttctctaccgcctcaagtga

SEQ ID NO: 112 - DNA, 2466 bp, Homo sapiens
atggacctcgcggcggcagcggagccgggcgccggcagccagcacctggaggtccgcgacgaggtggccgagaagtgccagaaactgttcctggacttcttggaggagtttcagagcagcgatggagaaattaaatacttgcaattagcagaggaactgattcgtcctgagagaaacacattggttgtgagttttgtggacctggaacaatttaaccagcaactttccaccaccattcaagaggagttctatagagtttacccttacctgtgtcgggccttgaaaacattcgtcaaagaccgtaaagagatccctcttgccaaggatttttatgttgcattccaagacctgcctaccagacacaagattcgagagctcacctcatccagaattggtttgctcactcgcatcagtgggcaggtggtgcggactcacccagttcacccagagcttgtgagcggaacttttctgtgcttggactgtcagacagtgatcagggatgtagaacagcagttcaaatacacacagccaaacatctgccgaaatccagtttgtgccaacaggaggagattcttactggatacaaataaatcaagatttgttgattttcaaaaggttcgtattcaagagacccaagctgagcttcctcgagggagtatcccccgcagtttagaagtaattttaagggctgaagctgtggaatcagctcaagctggtgacaagtgtgactttacagggacactgattgttgtgcctgacgtctccaagcttagcacaccaggagcacgtgcagaaactaattcccgtgtcagtggtgttgatggatatgagacagaaggcattcgaggactccgggcccttggtgttagggacctttcttataggctggtctttcttgcctgctgtgttgcgccaaccaacccaaggtttggggggaaagagctcagagatgaggaacagacagctgagagcattaagaaccaaatgactgtgaaagaatgggagaaagtgtttgagatgagtcaagataaaaatctataccacaatctttgtaccagcctgttccctactatacatggcaatgatgaagtaaaacggggtgtcctgctgatgctctttggtggcgttccaaagacaacaggagaagggacctctcttcgaggggacataaatgtttgcattgttggtgacccaagtacagctaagagccaatttctcaagcacgtggaggagttcagccccagagctgtctacaccagtggtaaagcgtccagtgctgctggcttaacagcagctgttgtgagagatgaagaatctcatgagtttgtcattgaggctggagctttgatgttggctgataatggtgtgtgttgtattgatgaatttgataagatggacgtgcgggatcaagttgctattcatgaagctatggaacagcagaccatatccatcactaaagcaggagtgaaggctactctgaacgcccggacgtccattttggcagcagcaaacccaatcagtggacactatgacagatcaaaatcattgaaacagaatataaatttgtcagctcccatcatgtcccgattcgatctcttctttatccttgtggatgaatgtaatgaggttacagattatgccattgccaggcgcatagtagatttgcattcaagaattgaggaatcaattgatcgtgtctattccctcgatgatatcagaagatatcttctctttgcaagacagtttaaacccaagatttccaaagagtcagaggacttcattgtggagcaatataaacatctccgccagagagatggttctggagtgaccaagtcttcatggaggattacagtgcgacagcttgagagcatgattcgtctctctgaagctatggctcggatgcactgctgtgatgaggtccaacctaaacatgtgaaggaagctttccggttactgaataaatcaatcatccgtgtggaaacacctgatgtcaatctagatcaagaggaagagatccagatggaggtagatgagggtgctggtggcatcaatggtcatgctgacagccctgctcctgtgaacgggatcaatggctacaatgaagacataaatcaagagtctgctcccaaagcctccttaaggctgggcttctctgagtactgccgaatctctaaccttattgtgcttcacctcagaaaggtggaagaagaagaggacgagtcagcattaaagaggagcgagcttgttaactggtacttgaaggaaatcgaatcagagatagactctgaagaagaacttataaataaaaaaagaatcatagagaaagttattcatcgactcacacactatgatcatgttctaattgagctcacccaggctggattgaaaggctccacagagggaagtgagagctatgaagaagatccctacttggtagttaaccctaactacttgctcgaagattga

SEQ ID NO: 113 - DNA, 813 bp, Homo sapiens
atgtggaacagtggattcgaaagctatggcagctcctcatacgggggagccggcggctacacgcagtccccggggggctttggatcgcccgcaccttctcaagccgaaaagaaatcaagagcccgagcccagcacattgtgccctgtactatatctcagctgctttctgccactttggttgatgaagtgttcagaattgggaatgttgagatttcacaggtcactattgtggggatcatcagacatgcagagaaggctccaaccaacattgtttacaaaatagatgacatgacagctgcacccatggacgttcgccagtgggttgacacagatgacaccagcagtgaaaacactgtggttcctccagaaacatatgtgaaagtggcaggccacctgagatcttttcagaacaaaaagagcctggtagcctttaagatcatgcccctggaggatatgaatgagttcaccacacatattctggaagtgatcaatgcacacatggtactaagcaaagccaacagccagccctcagcagggagagcacctatcagcaatccaggaatgagtgaagcagggaactttggtgggaatagcttcatgccagcaaatggcctcactgtggcccaaaaccaggtgttgaatttgattaaggcttgtccaagacctgaagggttgaactttcaggatctcaagaaccagctgaaacacatgtctgtatcctcaatcaagcaagctgtggattttctgagcaatgaggggcacatctattctactgtggatgatgaccattttaaatccacagatgcagaataa

SEQ ID NO: 114 - DNA, 942 bp, Homo sapiens
atgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttag

(19) United States
(12) Patent Application Publication
(10) Pub. No.: US 2014/0256735 A1
(43) Pub. Date: Sep. 11, 2014

(54) METHOD FOR ASSESSING A LIVER OF A PATIENT HAVING A CHRONIC HEPATITIS B VIRUS INFECTION

(71) Applicant: PURDUE RESEARCH FOUNDATION, West Lafayette, IN (US)

(72) Inventor: Ourania Andrisani, West Lafayette, IN (US)

(73) Assignee: PURDUE RESEARCH FOUNDATION, West Lafayette, IN (US)

(21) Appl. No.: 14/282,033

(22) Filed: May 20, 2014

Related U.S. Application Data
(63) Continuation-in-part of application No. PCT/US2012/070773, filed on Dec. 20, 2012.
(60) Provisional application No. 61/581,329, filed on Dec. 29, 2011.

(51) Int. Cl. C12Q 1/70

(52) U.S. Cl. CPC C12Q 1/70
USPC 514/249; 435/5

(57) ABSTRACT

The invention generally relates to method for assessing a liver of a patient having a chronic hepatitis B virus infection. In certain aspects, methods of the invention involve obtaining a sample from a patient having a chronic hepatitis B virus infection. An assay is conducted on the sample to determine a level of at least one biomarker regulated by Suz12/Znf198. The liver of the patient is then assessed based on the level of the at least one biomarker. Other aspects of the invention generally relate to reducing or eliminating replication of a hepatitis B virus, and preventing a chronic hepatitis B infection in a patient from progressing to a liver cancer.

### RELATED APPLICATION

The present application is a continuation-in-part of International patent application number PCT/US12/70773, filed Dec. 20, 2012, which claims the benefit of and priority to U.S. provisional application Ser. No. 61/581,329, filed Dec. 29, 2011, the content of each of which is incorporated by reference herein in its entirety.

### GOVERNMENT SUPPORT

This invention was made with government support under CA135192 and DK044533 awarded by National Institutes of Health. The government has certain rights in the invention.

### FIELD OF THE INVENTION

The invention generally relates to methods for assessing a liver of a patient having a chronic hepatitis B virus infection. Other aspects of the invention generally relate to reducing or eliminating replication of a hepatitis B virus, and preventing a chronic hepatitis B infection in a patient from progressing to a liver cancer.

### BACKGROUND

Hepatitis B virus is a virus that replicates in a liver of a patient. Chronic infection with hepatitis B virus either may be asymptomatic or may be associated with a chronic inflammation of the liver (chronic hepatitis). Chronic hepatitis B virus infection is a major factor in the pathogenesis of hepatocellular carcinoma (Beasley, et al., Lancet, 2:1129-1133, 1981; and Bruix et al., Cancer Cell, 5:215-219, 2004). Despite availability of a hepatitis B virus vaccine, the World Health Organization reports approximately 400 million people are chronically infected with hepatitis B virus.

Effective treatment regimens for chronic hepatitis B and hepatocellular carcinoma remain an important challenge (Zoulim, J Viral Hepatitis, 13:278-288, 2006, Llovet et al., N Engl J Med, 359:378-390, 2008; and Llovet et al., Hepatology, 48:1312-27, 2008). To reduce liver cancer, new biomarkers are needed for molecular classification and prognosis of hepatocellular carcinoma and for developing efficient therapies.

### SUMMARY

The invention generally relates to new biomarkers for assessing a liver of a patient having a chronic hepatitis B virus infection. The invention recognizes that infection by the hepatitis B virus results in down regulation of chromatin remodeling components Suz12/Znf198. Those components repress expression of certain proteins in the liver of normal patients. However, in the liver of patients having a chronic hepatitis B virus infection, proteins regulated by Suz12/Znf198 have increased expression, allowing those proteins to be used as biomarkers for determining whether a patient having a chronic hepatitis B virus infection will develop a liver cancer. Particularly, the expression pattern of certain biomarkers indicates that a chronic hepatitis B virus infection is progressing toward liver cancer prior to the appearance of any tumors in the liver. Other biomarker expression patterns indicate that presence of liver cancer.

In certain aspects, methods of the invention involve obtaining a sample from a patient having a chronic hepatitis B virus infection. The sample may be any clinically acceptable tissue or body fluid sample, such as blood, urine, saliva, sputem, stool, puss, aspirate, or biopsied tissue. In certain embodiments, the sample is liver tissue, typically obtained from a biopsy. Methods of the invention further involve conducting an assay on the sample to determine a level of at least one biomarker regulated by Suz12/Znf198. The liver of the patient is then assessed based on the level of the at least one biomarker. In certain embodiments, prior to obtaining the sample, the method further involves determining that the patient has a chronic hepatitis B virus infection. In other embodiments, methods of the invention further involve providing a course of treatment to the patient based on results of the assessing step. The treatment plan will be based on the status of the liver of the patient. For example, the course of treatment may involve continued monitoring or administration of anti-viral or chemotherapeutic drugs.

Methods of the invention may make use of a single biomarker or a combination of biomarkers that are regulated by Suz12/Znf198 and have differential expression between normal patients and patients having a chronic hepatitis B virus infection. Exemplary biomarkers regulated by Suz12/Znf198 include BAMBI, CCND2, DKK2, DLK1, EpCAM, and IGFII. Methods of the invention may be conducted with any one of those exemplary biomarkers or may use any combination of those biomarkers. In particular embodiments, the biomarkers are used to grade the progression of a chronic hepatitis B virus infection toward a liver cancer. For example, overexpression of at least one of CCND2, EpCAM, or IGFII indicates chronic hepatitis B virus infection progressing toward liver cancer. In particular embodiments, the overexpression of all three biomarkers indicates chronic hepatitis B virus infection progressing toward liver cancer.

In other embodiments, the assay also detects a level of at least one proliferation biomarker. Exemplary proliferation biomarkers include PLK1, CCNA1, MCM4-6, RPA2, and TYMS. Methods of the invention may be conducted with any one of or a combination of the proliferative biomarkers in combination with the Suz12/Znf198 regulated biomarkers. In particular embodiments, the proliferative biomarkers are used in combination with the Suz12/Znf198 regulated biomarkers to grade the progression of a chronic hepatitis B virus infection toward a liver cancer. For example, overexpression of at least one of BAMBI, EpCAM, or PLK1 in combination with the expression levels of the Suz12/Znf198 regulated biomarkers indicates chronic hepatitis B virus infection progressing toward liver cancer. In certain embodiments, the overexpression of all three proliferative biomarkers in combination with the expression levels of the Suz12/Znf198 regulated biomarkers indicates chronic hepatitis B virus infection progressing toward liver cancer. In certain embodiments, the proliferative biomarker PLK1 is used in combination with the Suz12/Znf198 regulated biomarkers.

Methods of the invention may also determine aggressiveness of the liver cancer based upon which biomarker or combination of biomarkers are overexpressed. For example, overexpression of a combination of DKK1, DLK1, IGFII, and EpCAM indicates an aggressive form of liver cancer.

Another aspect of the invention provides methods for reducing or eliminating replication of a hepatitis B virus, particularly, in a patient suffering from a chronic hepatitis B virus infection. The invention recognizes that PLK1 plays a critical role in the replication of the hepatitis B virus. Accordingly, administering a PLK1 inhibitor to a patient reduces or eliminates replication of the hepatitis B virus. Reducing or eliminating replication of the hepatitis B virus treats the chronic hepatitis B virus infection in the patient. Generally, the PLK1 inhibitor is formulated with a pharmaceutically acceptable carrier. The PLK1 inhibitor may also be provided as a unitary dose.

Other aspects of the invention relate to methods for preventing a chronic hepatitis B infection in a patient from progressing to a liver cancer. Those methods involve determining that a patient has a chronic hepatitis B infection that is progressing toward a liver cancer. The methods further involve administering a PLK1 inhibitor to thereby reduce or eliminate replication of a hepatitis B virus, thereby preventing the chronic hepatitis B infection in the patient from progressing to a liver cancer.

### BRIEF DESCRIPTION OF THE DRAWINGS

FIGS. 1A-C show increased protein levels of Plk1 and reduced levels of Znf198 and Suz12 during liver cancer pathogenesis in X/c-myc bitransgenics. FIG. 1A shows immunoblots of liver lysates from wild type and X/c-myc bitransgenics at the indicated ages employing antibodies for Plk1, Znf198, Suz12 and actin. A representative immunoblot is shown from at least three independent whole cell extract preparations. Protein levels of Plk1, Znf198 and Suz12 were quantified vs. actin by ImageJ software. FIG. 1B shows quantification of protein levels of Plk1, Znf198 and Suz12, in liver lysates from 2-week and 4-month WT and X/c-myc bitransgenic mice, is the average of values from all tested animals in each group, from at least three independent whole cell extract preparations. Error bars indicate+/−standard error of the mean. * indicates p<0.01 (student's t test). FIG. 1C shows quantification of protein levels of Plk1, Znf198 and Suz12, in liver lysates from individual 12-month X/c-myc bitransgenics, in paired tumor vs. peri-tumoral tissue. The average from at least three independent experiments is shown. Error bars indicate+/−standard error of the mean. * indicates p<0.01 and ** p<0.05 (student's t test).

FIGS. 2A-C show enhanced expression and activation of Plk1 in X/c-myc bitransgenics. FIG. 2A shows real-time PCR quantification of X mRNA, employing liver tissue from the indicated X/c-myc bitransgenics. Results are from three independent RNA preparations, analyzed by real-time PCR in triplicates with GAPDH as internal control. FIG. 2B shows immunoblots of liver lysates from 4-month wild type, X and cmyc monotransgenic mice, and X/c-myc bitransgenics, employing indicated antibodies. A representative experiment is shown from two independent WCE preparations. FIG. 2C shows quantification of indicated proteins from immunoblots shown in FIG. 2B. vs. actin is by ImageJ software, from two independent WCE preparations. Error bars indicate+/−standard error of the mean.

FIG. 3 shows increased protein levels of Plk1 and reduced levels of Suz12 1 in hepatic tumors from woodchuck hepatitis virus-infected animals. Immunoblots of Plk1, Suz12 and actin using lysates from woodchuck hepatitis virus-induced liver tumors. Tumors were classified as well-differentiated HCC (T1), less-differentiated (T2), and least-differentiated (T3) based on anaplastic features and tumor size (Jacob et al., Hepatology, 39:1008-1016, 2004). Relative intensity of indicated protein bands was quantified vs. actin by ImageJ software. A representative assay is shown from at least three independent whole cell extract preparations.

FIGS. 4A-B show identification of Suz12/PRC2-repressed genes in untransformed hepatocytes. FIG. 4A shows quantification by real-time PCR of Suz12/PRC2 target genes using total RNA isolated from pXtransformed cells (Studach et al., Hepatology, 50:414-423, 2009), 4pX-1-Suz12^{kd} (Wang et al., Hepatology, 53:1137-47, 2011) and 4pX-1GIPZ 10 cells (Wang et al., Hepatology, 53:1137-47, 2011) expressing pX by tetracycline removal for 24 h. Results are from three independent RNA preparations, analyzed by real-time PCR in triplicates with GAPDH as internal control. Error bars indicate+/−standard error of the mean. FIG. 4B shows chromatin immunoprecipitation (ChIP) assays with Suz12 antibody in 4pX-1^{GIPZ} cells, pX-transformed, and 4pX-1-SUZ12^{kd} 14 cells. ChIP-derived DNA was amplified by PCR with primer pairs (See Table 1) for indicated genes. Real-time PCR quantification of indicated genes is expressed as percent reduction in Suz12 occupancy in pXtransformed and 4pX-1-Suz12^{kd} cells relative to untransformed 4pX-1^{GIPZ} 17 cells. Error bars indicate+/−standard error of the mean.

FIGS. 5A-B show increased expression of Suz12-repressed genes in the context of Znf198 knockdown. FIG. 5A shows quantification by real-time PCR of Suz12-repressed genes using total RNA isolated from pXtransformed cells (Studach et al., Hepatology, 50:414-423, 2009), 4pX-1-Znf198^{kd} (Wang et al., Hepatology, 53:1137-47, 2011) and 4pX-1^{GIPZ} 22 cells (Wang et al., Hepatology, 53:1137-47, 2011) expressing pX by tetracycline removal for 24 h. Results are from three independent RNA preparations, analyzed by 24 real-time PCR in triplicates with GAPDH as internal control. Error bars indicate+/−standard error of the mean. FIG. 5B shows chromatin immunoprecipitation (ChIP) assays 1 with Suz12 antibody in 4pX-1^{GIPZ} cells, pX-transformed and 4pX-1-Znf198^{kd} cells. ChIP-derived DNA was amplified by PCR with primer pairs (see Table 1) for indicated genes. Real-time PCR quantification of indicated genes is expressed as percent reduction in Suz12 occupancy in pXtransformed and 4pX-1-Znf198^{kd} cells relative to untransformed 4pX-1^{GIPZ} cells. Error bars indicate+/−standard error of the mean.

FIGS. 6A-B show expression of the Suz12 repressed genes during distinct stages of hepatocellular carcinoma pathogenesis in X/c-myc bitransgenics. Quantification by real-time PCR of the indicated Suz12 repressed genes in FIG. 6A, and the proliferation gene cluster in FIG. 6B, using total RNA isolated from liver of X/c-myc bitransgenics at the indicated stages of hepatocellular carcinoma pathogenesis. Total RNA isolated from liver tissues shown in FIG. 1A, was pooled and analyzed by real-time PCR. Each PCR reaction was performed in triplicates from three independent RNA preparations. Quantification is relative to GAPDH used as internal control. Error bars indicate+/−standard error of the mean.

FIGS. 7A-D show enhanced expression of the Suz12 repressed genes and proliferation genes in the presence of hepatitis B virus replication. FIG. 7A shows quantification of hepatitis B virus replication in HepAD38 cells that support hepatitis B virus replication following removal of tetracycline (Ladner et al., Antimicrobial Agents and Chemotherapy, 41:1715-1720, 1997). HepAD38 cells were grown with (+) or without (−) tetracycline for 10 days, i.e. without (−) or with (+) hepatitis B virus replication, respectively. Hepatitis B virus genome equivalents were quantified by real-time PCR of DNA isolated from purified intracellular virions as described (Wang et al., Hepatology, 53:1137-47, 2011). (Left panel) Effect of DMSO addition (5.0 μl) on hepatitis B virus replication. (Right panel) 250 nM or 500 nM BI 2536 dissolved in DMSO (5.0 μl) added on day 8 for 48 h or day 9 for 24 h, as indicated, prior to cell harvesting. Results represent the average of three independent experiments. Error bars indicate+/−standard 1 error of the mean. FIG. 7B shows immunoblots of Suz12 and Znf198 using lysates of HepAD38 cells grown with (+) or without (−) tetracycline for 10 days and treated for 24 h prior to harvesting with 500 nM BI2536. Relative intensity of indicated protein levels was quantified by ImageJ software vs. actin. A representative experiment is shown from three independent experiments. FIG. 7C shows quantification by real-time PCR of the proliferation gene cluster. FIG. 7D shows quantification of Suz12 repressed genes using total RNA isolated from HepAD38 cells grown with (+) or without (−) tetracycline for 10 days and treated for 24 h prior to harvesting with 500 nM BI2536. Quantification is relative to GAPDH used as internal control. Results are from three independent RNA isolations. PCR assays were performed in triplicates. Error bars indicate+/−standard error of the mean.

FIGS. 8A-B show enhanced expression of the proliferation gene cluster and the Suz12 repressed genes in hepatic tumors from woodchuck hepatitis virus infected animals. Tumors were classified (Jacob et al., Hepatology, 39:1008-1016, 2004) as well-differentiated hepatocellular carcinoma (T1), less-differentiated (T2), and least-differentiated (T3). Total RNA isolated from individual paired liver tissues, tumor vs. peri-tumoral, shown in FIGS. 2A-B, was analyzed by real-time PCR. FIG. 8A. shows quantification of the proliferation gene cluster. FIG. 8B shows quantification of the Suz12 repressed genes. Quantification is relative to GAPDH used as internal control. Results are the average of three independent experiments, each PCR assay performed in triplicates. The fold induction of each gene with standard error of the mean is shown in Table 4.

FIG. 9 shows chromatin immunoprecipitation (ChIP) assays with Suz12 antibody or IgG as indicated in 4pX-1 cells, pX-transformed cells (Studach et al., Hepatology, 50:414-423, 2009), and 4pX-1-Suz12^{kd} cells (Wang et al., Hepatology, 53:1137-47, 2011). The ChIP-derived DNA was amplified by PCR with primer pairs (see Table 1) for indicated genes and PCR products were analyzed by agarose gel electrophoresis.

FIGS. 10A-C show that HepAD38 cells that support hepatitis B virus replication following removal of tetracycline (Ladner et al., Antimicrobial Agents and Chemotherapy, 41:1715-1720, 1997), were grown with (+) or without (−) tetracycline for 10 days, i.e. without (−) or with (+) hepatitis B virus replication, respectively. FIG. 10A shows that 250 nM or 500 nM BI 2536 was added on day 8 for 48 h or day 9 for 24 h, as indicated, prior to cell harvesting. Whole cell extract isolated without (−) or with (+) 1 addition of BI 2536 were immunoblotted for hepatitis B virus core antigen, caspase 3 and cleaved/active caspase. FIG. 10B shows that 250 nM or 500 3 nM BI 2536 was added on day 8 for 48 h or day 9 for 24 h, as indicated, prior to isolation of total RNA. Expression of viral RNA was quantified by real-time PCR employing primer pairs described by Zhang et al. (Hepatology 53:1476-1485, 2011). Results are the average from two independent WCE preparations. Error bars indicate+/−standard error of the mean. FIG. 10C shows quantification of protein levels of Suz12 and Znf198 from immunoblots shown in FIG. 5B, using lysates of HepAD38 cells grown with (+) or without (−) tetracycline for 10 days and treated for 24 h prior to harvesting with 500 nM BI2536. Quantification of indicated proteins is by ImageJ software vs. actin. Results are the average from two independent WCE preparations. Error bars indicate+/−standard error of the mean.

FIG. 11 shows the nucleic acid sequence of human Plk1 (SEQ ID NO.: 99).

FIGS. 12A-B show a first nucleic acid coding sequence of human Znf198 (SEQ ID NO.: 100).

FIGS. 13A-C show a second nucleic acid coding sequence of human Znf198 (SEQ ID NO.: 101).

FIGS. 14A-B show the nucleic acid sequence of human Suz12 (SEQ ID NO.: 102).

FIG. 15 shows the nucleic acid sequence of human BAMBI (SEQ ID NO.: 103).

FIG. 16 shows the nucleic acid sequence of human CCND2 (SEQ ID NO.: 104).

FIG. 17 shows the nucleic acid sequence of human DKK2 (SEQ ID NO.: 105).

FIG. 18 shows the nucleic acid sequence of human DLK1 (SEQ ID NO.: 106).

FIG. 19 shows the nucleic acid sequence of human EpCAM (SEQ ID NO.: 107).

FIG. 20 shows the nucleic acid sequence of human IGFII (SEQ ID NO.: 108).

FIG. 21 shows the nucleic acid sequence of human CCNA1 (SEQ ID NO.: 109).

FIGS. 22A-B show the nucleic acid sequence of human MCM4 (SEQ ID NO.: 110).

FIGS. 23A-B show the nucleic acid sequence of human MCM5 (SEQ ID NO.: 111).

FIGS. 24A-B shows the nucleic acid sequence of human MCM6 (SEQ ID NO.: 112).

FIG. 25 shows the nucleic acid sequence of human RPA2 (SEQ ID NO.: 113).

FIG. 26 shows the nucleic acid sequence of human TYMS (SEQ ID NO.: 114).

### DETAILED DESCRIPTION

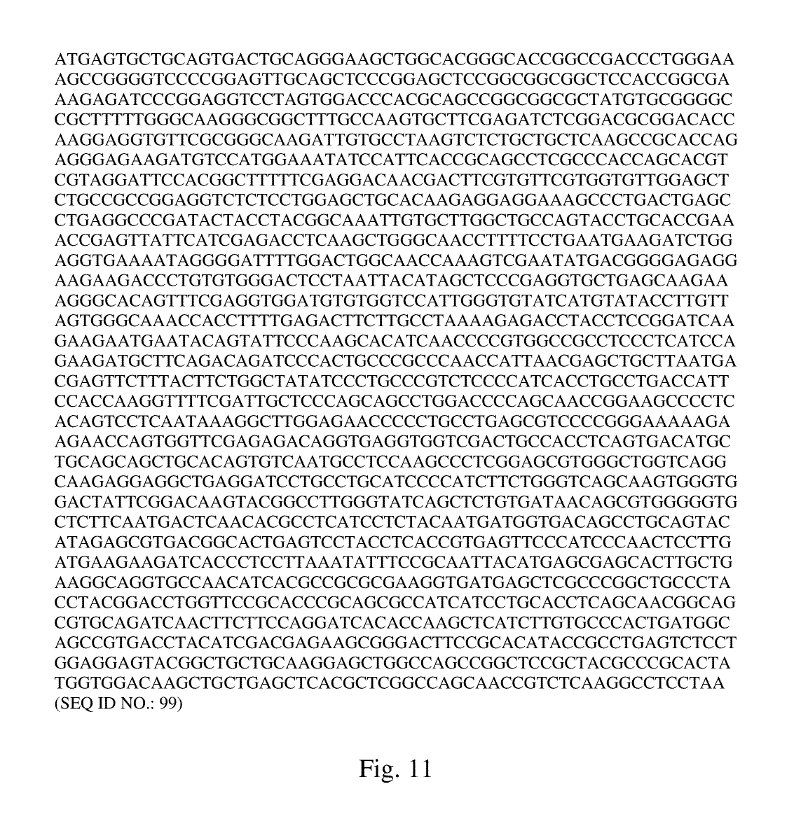

Pathogenesis of hepatitis B virus-mediated hepatocellular carcinoma involves chronic liver inflammation (Hagen et al., Proc Natl Acad Sci USA, 91:12808-12812, 1994)) and effects of the weakly oncogenic hepatitis B virus X protein (pX) (Terradillos et al., Oncogene, 14:395-404, 1997; and Madden et al., J Virol, 75:3851-3858, 2001). In a cell culture model of non-transformed hepatocytes (Tarn et al., J Biol Chem, 274:2327-2336, 1999), pX activates cellular mitogenic pathways (Lee et al., J Biol Chem, 277:8730-8740, 2002; and Wang et al., J Biol Chem, 283:25455-25467, 2008), promotes DNA damage induced by DNA re-replication (Rakotomalala et al., J Biol Chem, 283:28729-28740, 2008), and activates Polo like kinase 1 (Plk1; Studach et al., J Biol Chem, 285:30282-93, 2010). In turn, activated Plk1 mediates checkpoint adaptation, generating partial polyploidy and transformation (Studach et al., J Biol Chem, 285:30282-93, 2010; and Studach et al., Hepatology, 50: 414-423, 2009). Significantly, Plk1 is elevated in liver tumors from chronic hepatitis B virus patients (Chen et al., Mol Biol Cell, 13:1929-1939, 2002; and Wang et al., Hepatology, 53:1137-47, 2011). The nucleic acid sequence of Plk1 is shown in FIG. 11.

Likely substrates of Plk1 relevant to hepatitis B virus-mediated hepatocellular carcinoma pathogenesis are proteins zinc finger (Znf198) and suppressor of zeste 12 homolog (Suz12) which are involved both in pX-mediated transformation and hepatitis B virus replication.

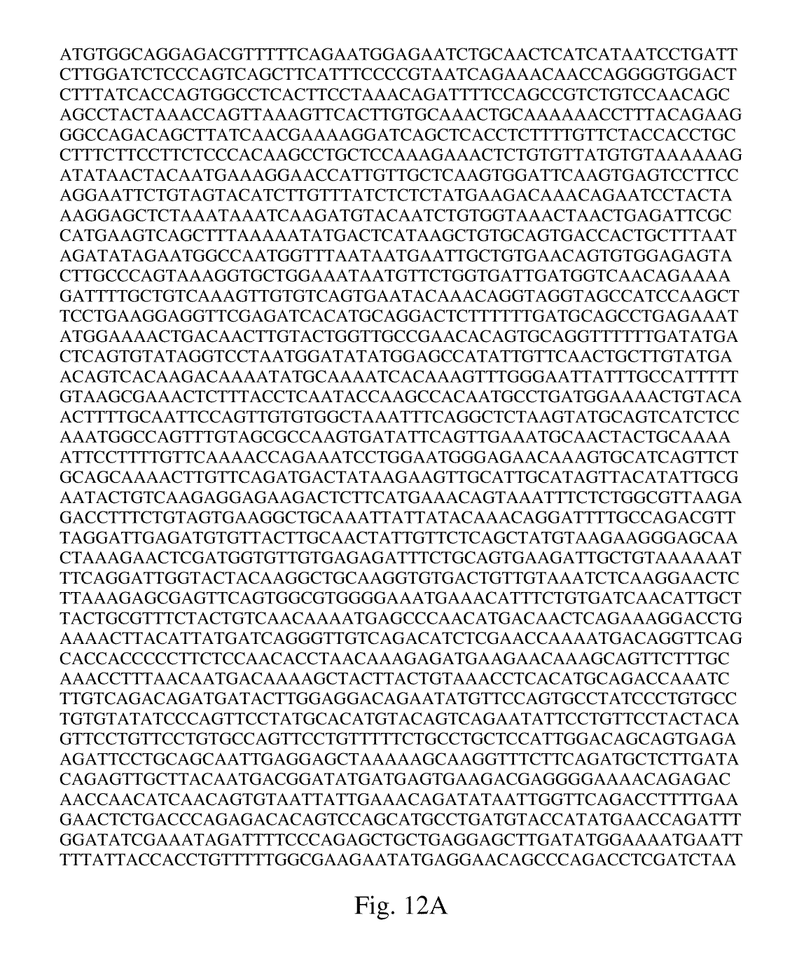

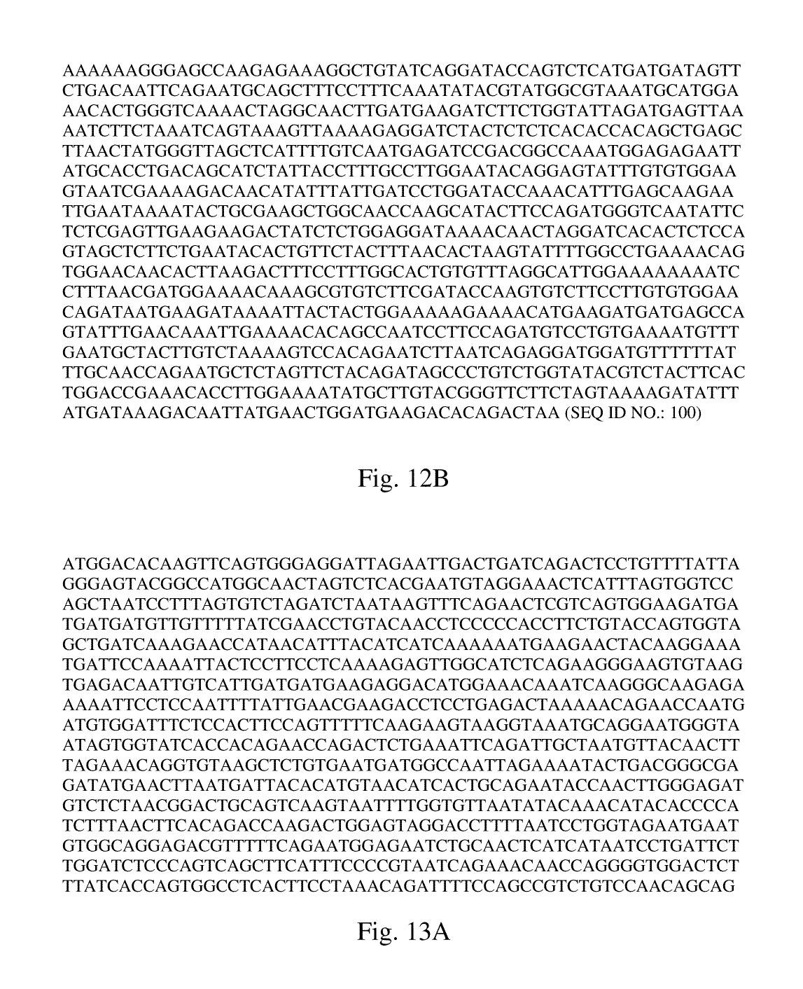

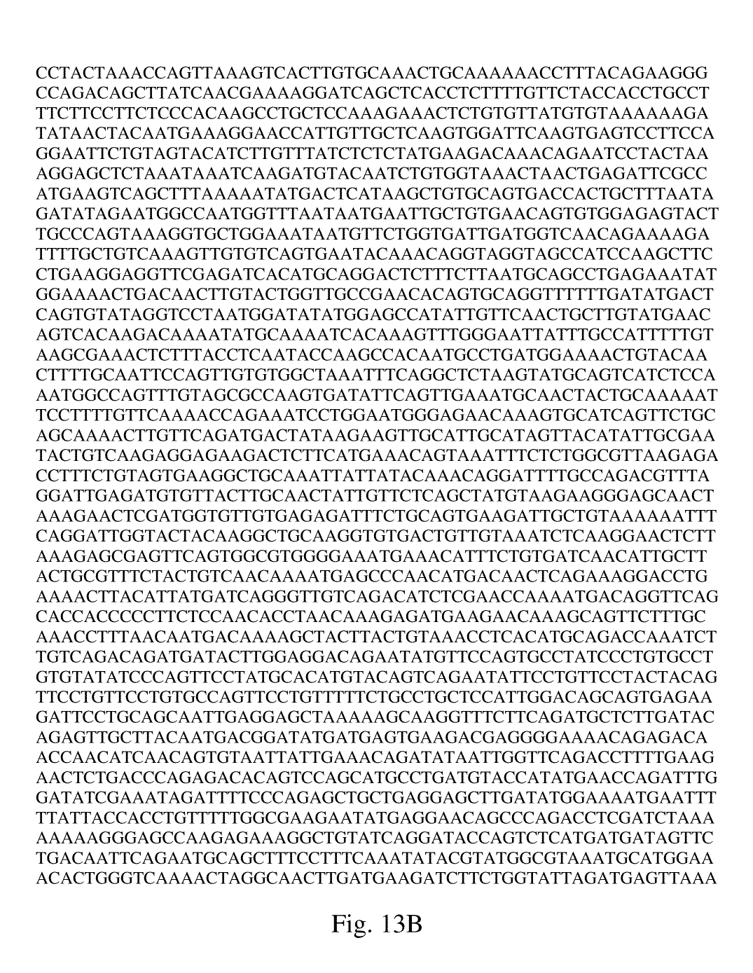

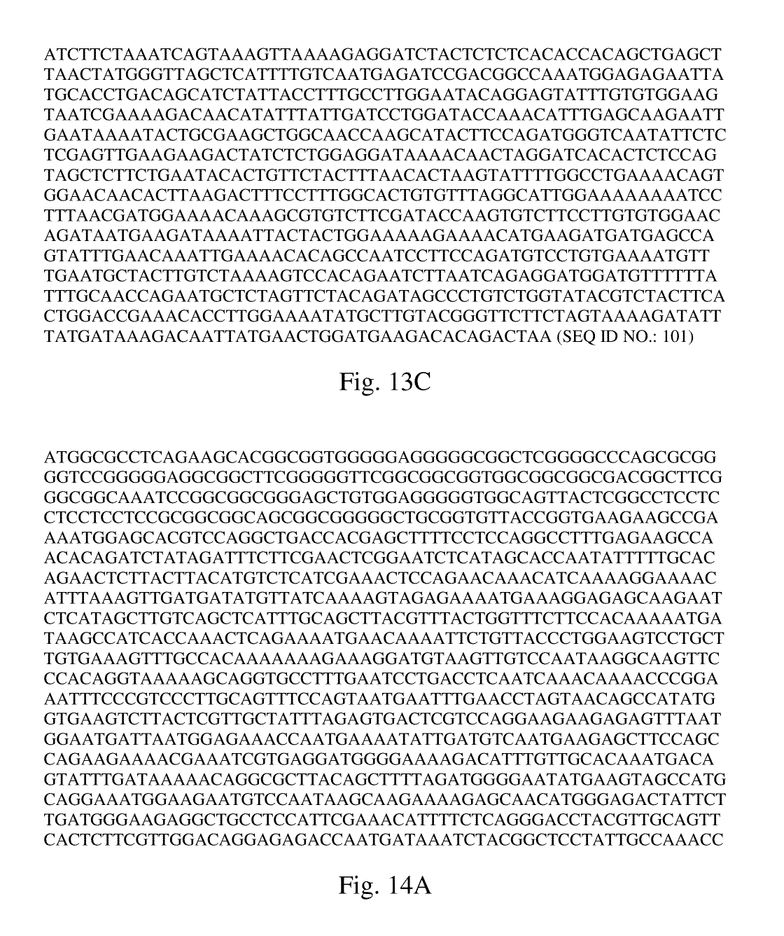

Znf198 stabilizes the LSD1-CoREST-HDAC1 co-repressor complex that removes 16 histone modifications associated with transcriptional activation (Gocke et al., ZNF198 stabilizes the LSD-1-CoREST-HDAC1 complex on 16 chromatin through its MYM-type zinc fingers, PLoS ONE, 3:e3255, 2008). In addition, the ZNF198 gene, located on chromosome 13q12.11, is frequently affected by loss of heterozygosity in early onset hepatocellular carcinoma and correlates with high tumor grade in hepatitis B virus-induced tumors (Moinzadeh et al., British J Cancer, 92:935-941, 2005; and Chen et al., Genes Chromosomes & Cancer, 44:320-328, 2005). Znf198 has two coding sequences. The first nucleic acid coding sequence of Znf198 is shown in FIG. 12, and the second nucleic acid coding sequence of Znf198 is shown in FIG. 13.

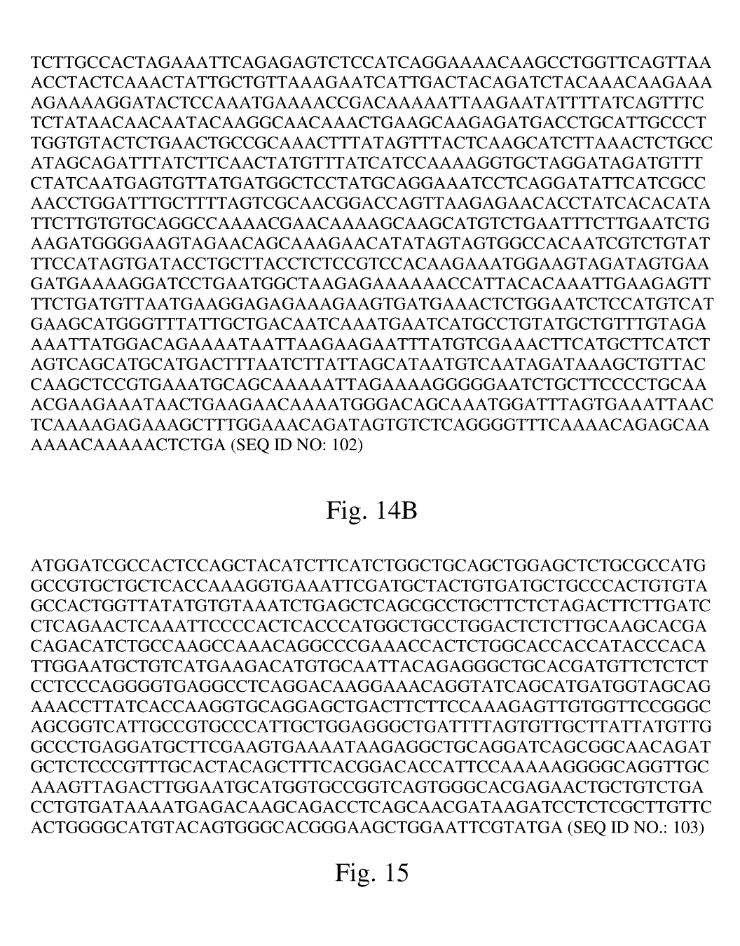

Suz12 is an essential component of the Polycomb Repressive chromatin remodeling Complex 2 (PRC2) that mediates the repressive trimethylation of H3 on Lys27, H3K27me3, (Squazzo et al., Genome Res, 16:890-900, 2006; Simon et al., Nature Reviews Mol Cell Biol, 10:697-708, 2009; and Villa et al., Cancer Cell, 11:513-525, 2007). The nucleic acid sequence of Suz12 is shown in FIG. 14.

In human embryonic fibroblasts, more than 1000 genes are transcriptionally silenced by PRC2 (Bracken et al., Genes Dev, 209:1123-1136, 2006). A subset of Suz12/PRC2 target genes that include EpCAM, IGFII, DKK1,2, DLK1, MYC, MCM5, RPA2, CCNA1 and CCND2 are overexpressed in human hepatocellular carcinomas, based on microarray studies of human liver tumors. EpCAM and DLK1 are normally expressed in hepatoblasts (Andrisani et al., Seminars Cancer Biol, 21:4-9, 2011; and De Boer et al., J Pathol, 188:201-206, 1999) and together with DKK1,2 and MYC are up-regulated in hepatic cancer initiating/stem cells (Yamashita et al., Gastroenterology, 136:1012-1024, 2009; and Terris et al., J Hepatol, 52:280-291, 2010) and hepatocellular carcinomas of poor prognosis derived from chronically hepatitis B virus-infected patients (Yamashita et al., Cancer Res, 68:1451-1461, 2008; Boyault et al., Hepatology, 45:42-52, 2007; and Breuhahn et al., Cancer Res, 64:6058-6064, 2004). Furthermore, MYC, MCM5, RPA2, CCNA1 and CCND2, the proliferation gene cluster, are also over-expressed in such hepatocellular carcinomas (Chen et al., Mol Biol Cell, 13:1929-1939, 2002; Yamashita et al., Gastroenterology, 136:1012-1024, 2009; Terris et al., J Hepatol, 52:280-291, 2010; Yamashita et al., Cancer Res, 68:1451-1461, 2008; Boyault et al., Hepatology, 45:42-52, 2007; Breuhahn et al., Cancer Res, 64:6058-6064, 2004; and Lee et al., Hepatology, 40:667-676, 2004). These observations suggest deregulation of PRC2 activity is involved in hepatocellular carcinoma pathogenesis.

PRC2 interacts with long noncoding RNAs and short RNAs transcribed from the 5′ end of PRC2 repressed genes (Zhao et al., Molecular Cell, 40:939-953, 2010; and Kanhere et al., Cell; 38:675-688, 2010). These PRC2/RNA interactions suggest mechanisms of PRC2-mediated regulation of gene expression. For example, PRC2 regulates expression of the imprinted DLK1 gene in mouse ESCs via direct interactions with the noncoding RNA Gtl2 acting as cofactor. DLK1 is expressed in hepatic progenitors and hepatoblasts and overexpressed in hepatic cancer stem cells from hepatitis B virus-infected patients with hepatocellular carcinoma. More interestingly, hepatitis B virus-mediated liver tumors associated with EpCAM have been identified with positivity and poor prognosis (C3 type), enhanced expression of a cluster of microRNAs encoded by the imprinted DLK1-DIO3 region on chromosomes (Toffanin et al., MicroRNA-Based Classification of Hepatocellular Carcinoma and Oncogenic Role 18 of miR-517a. Gastroenterology, in press). Together, these observations suggest loss of PRC2 activity is involved in hepatitis B virus-mediated hepatocarcinogenesis.

The invention recognizes that there is down-regulation of the PRC2 component Suz12 during pX-mediated transformation and hepatitis B virus replication in vitro. Methods of the invention are based on data herein that show that genes repressed by the Suz12/PRC2 complex are activated during hepatitis B virus mediated hepatocarcinogenesis and hepatitis B virus replication. The data were obtained by examining expression of hepatocellular carcinoma-relevant Suz12 target genes during pX-mediated transformation and hepatocellular carcinoma pathogenesis, as well as in the context of hepatitis B virus replication.

In certain aspects, methods of the invention involve obtaining a sample from a patient having a chronic hepatitis B virus infection. The sample is typically a tissue or body fluid that is obtained in any clinically acceptable manner. A tissue is a mass of connected cells and/or extracellular matrix material, e.g. skin tissue, endometrial tissue, nasal passage tissue, CNS tissue, neural tissue, eye tissue, liver tissue, kidney tissue, placental tissue, mammary gland tissue, placental tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sweat, amniotic fluid, menstrual fluid, mammary fluid, follicular fluid of the ovary, fallopian tube fluid, peritoneal fluid, urine, and cerebrospinal fluid (CSF), such as lumbar or ventricular CSF. A sample may also be a fine needle aspirate or biopsied tissue. A sample also may be media containing cells or biological material. A sample may also be a blood clot, for example, a blood clot that has been obtained from whole blood after the serum has been removed. In particular embodiments, the sample is liver tissue, typically obtained from a biopsy.

Nucleic acid is extracted from the sample according to methods known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a genomic sample is collected from a subject followed by enrichment for genes or gene fragments of interest, for example by hybridization to a nucleotide array. The sample may be enriched for genes of interest (e.g., genes regulated by Suz12/Znf198) using methods known in the art, such as hybrid capture. See for examples, Lapidus (U.S. Pat. No. 7,666,593), the content of which is incorporated by reference herein in its entirety.

RNA may be isolated from eukaryotic cells by procedures that involve lysis of the cells and denaturation of the proteins contained therein. Tissue of interest includes liver cells. RNA may be isolated from fluids of interest by procedures that involve denaturation of the proteins contained therein. Fluids of interest include blood. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX (see Ausubel et al., CURRENT PROTOCOLS IN MOLECULAR BIOLOGY, vol. 2, Current Protocols Publishing, New York (1994). Once bound, poly(A)+mRNA is eluted from the affinity column using 2 mM EDTA/0.1% SDS.

If desired, a protein from the sample to be analyzed can easily be isolated using techniques which are well known to those of skill in the art. Protein isolation methods can, for example, be such as those described in Harlow and Lane, 1988, Antibodies: A Laboratory Manual, Cold Spring Harbor Laboratory Press (Cold Spring Harbor, N.Y.), which is incorporated by reference herein in its entirety.

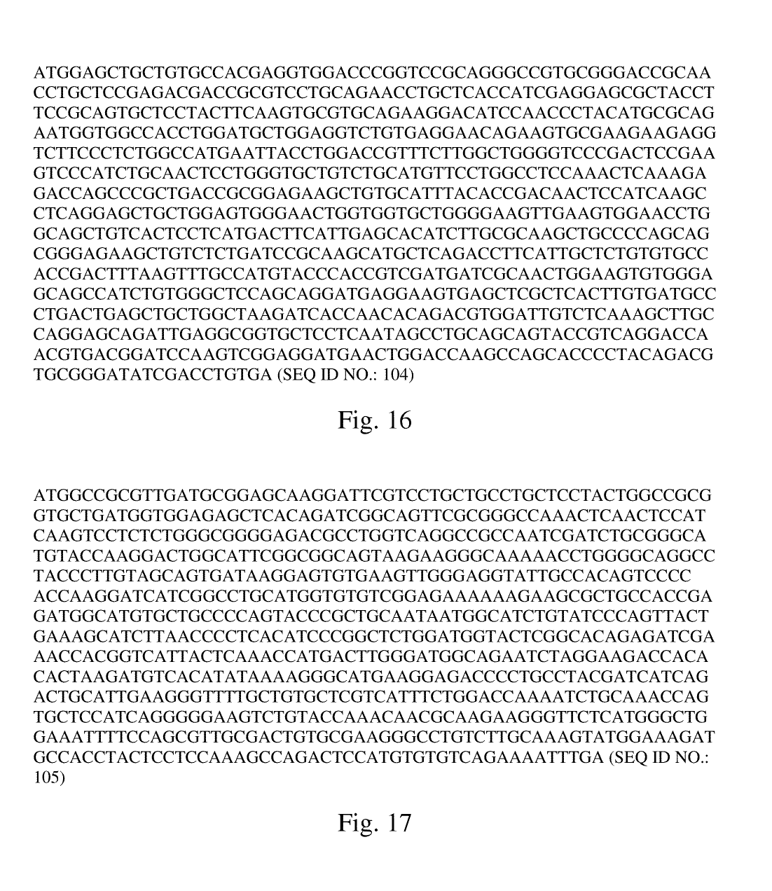

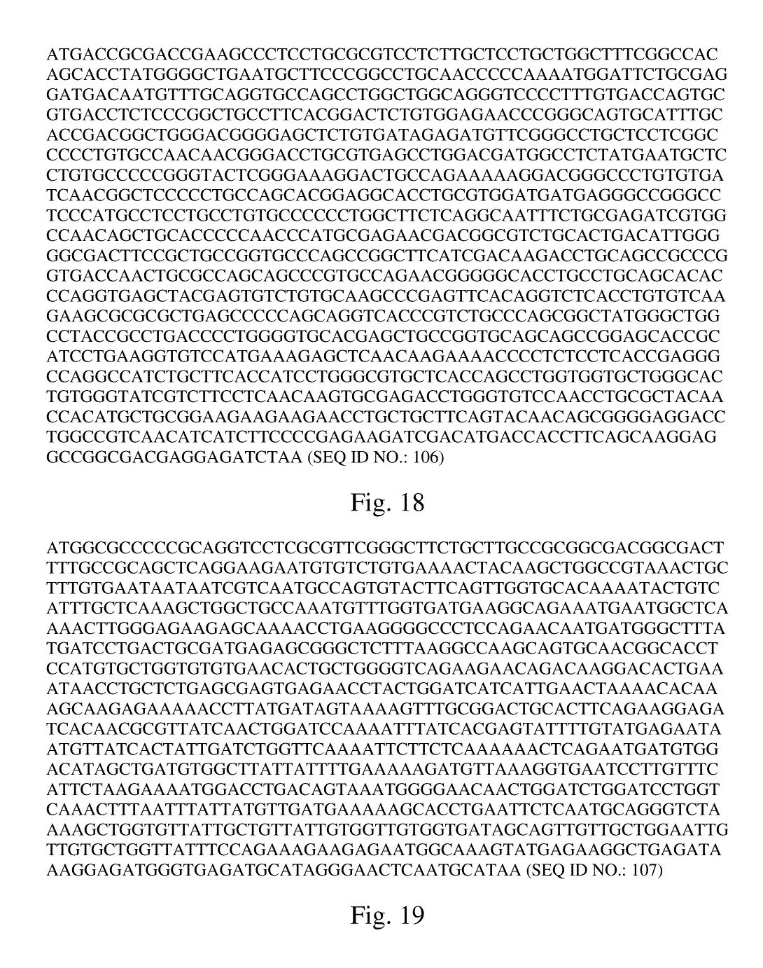

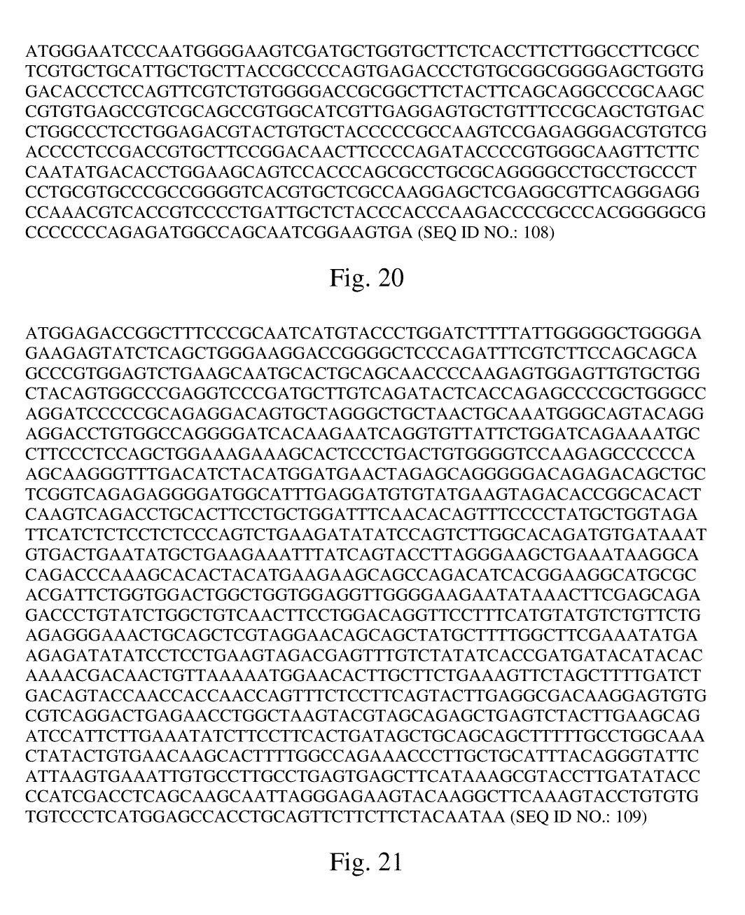

An assay is conducted on the sample to determine a level of at least one biomarker regulated by Suz12/Znf198. The liver of the patient is then assessed based on the level of the at least one biomarker. A biomarker generally refers to a molecule that may act as an indicator of a biological state. Biomarkers for use with methods of the invention may be any marker that is associated with Suz12/Znf198 and is differentially expressed between normal patients and patients having a chronic hepatitis B virus infection. Exemplary biomarkers include genes and gene products (e.g., RNA and protein). Exemplary biomarkers regulated by Suz12/Znf198 include BAMBI (nucleic acid sequence shown in FIG. 15), CCND2 (nucleic acid sequence shown in FIG. 16), DKK2 (nucleic acid sequence shown in FIG. 17), DLK1 (nucleic acid sequence shown in FIG. 18), EpCAM (nucleic acid sequence shown in FIG. 19), and IGFII (nucleic acid sequence shown in FIG. 20). Methods of the invention may be conducted with any one of those exemplary biomarkers or may use any combination of those biomarkers. In particular embodiments, the biomarkers are used to grade the progression of a chronic hepatitis B virus infection toward a liver cancer. For example, overexpression of at least one of CCND2, EpCAM, or IGFII indicates chronic hepatitis B virus infection progressing toward liver cancer. In particular embodiments, the overexpression of all three biomarkers indicates chronic hepatitis B virus infection progressing toward liver cancer.

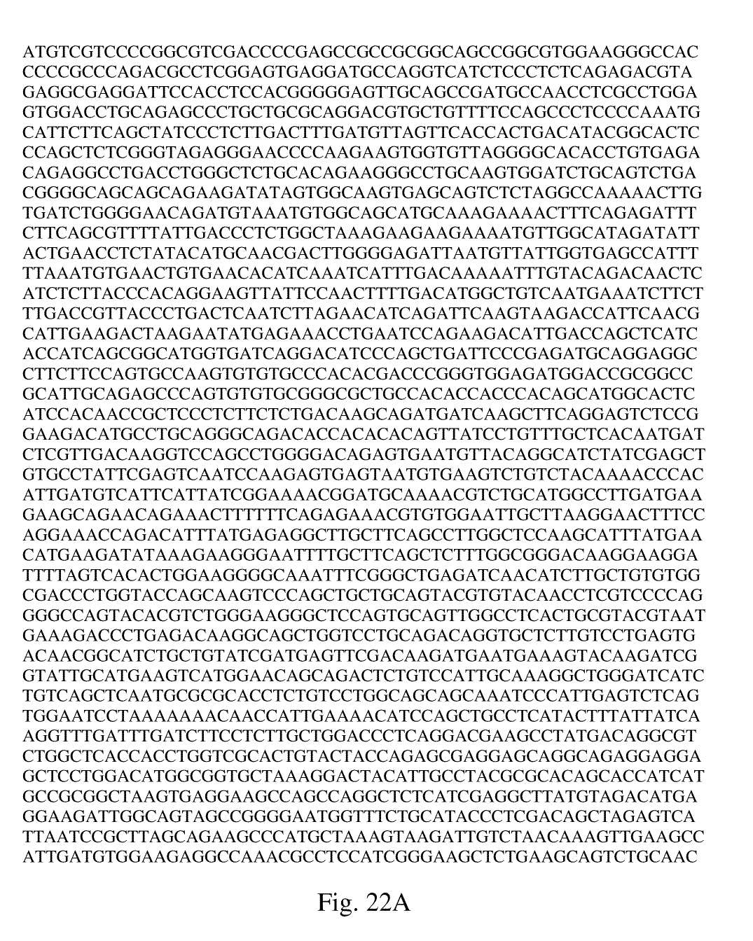

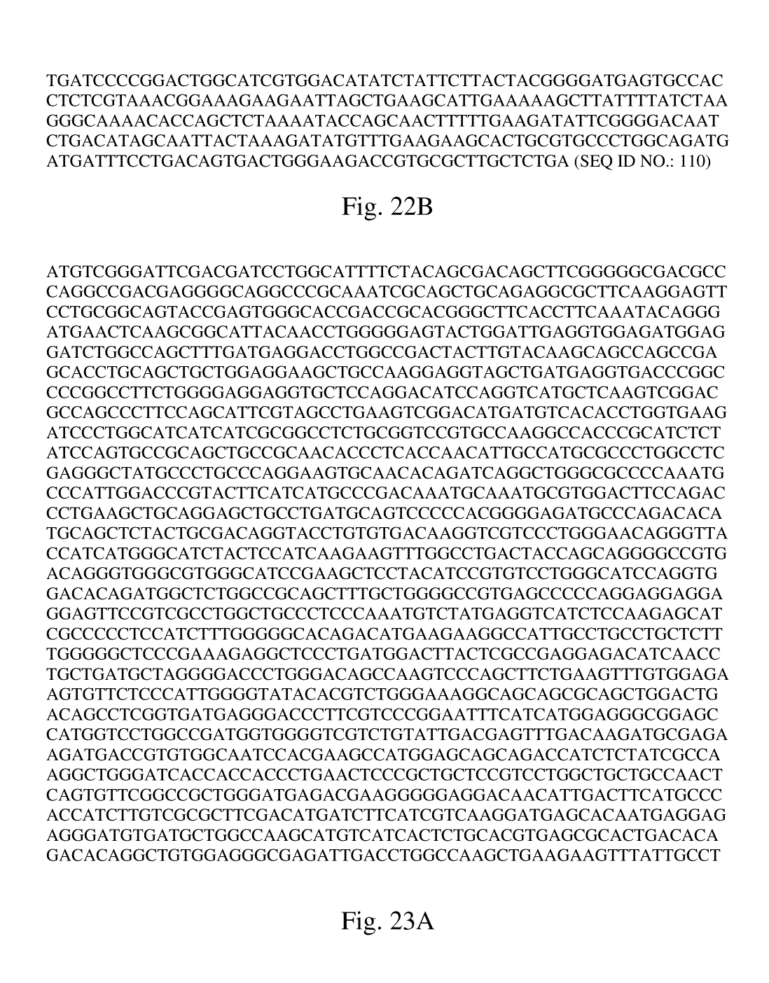

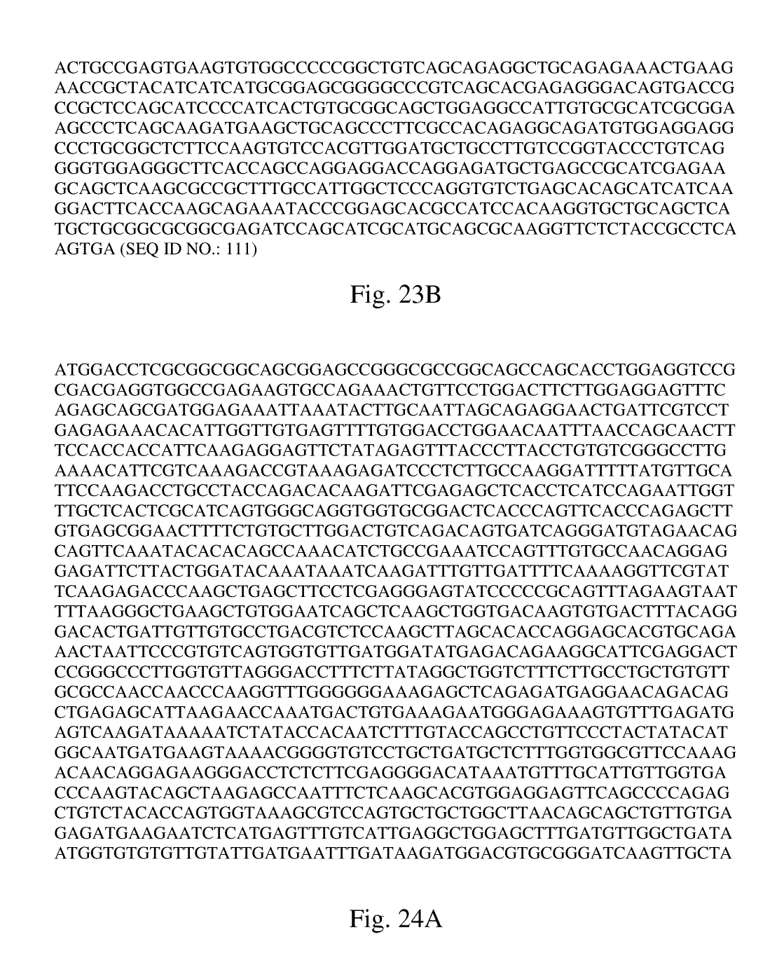

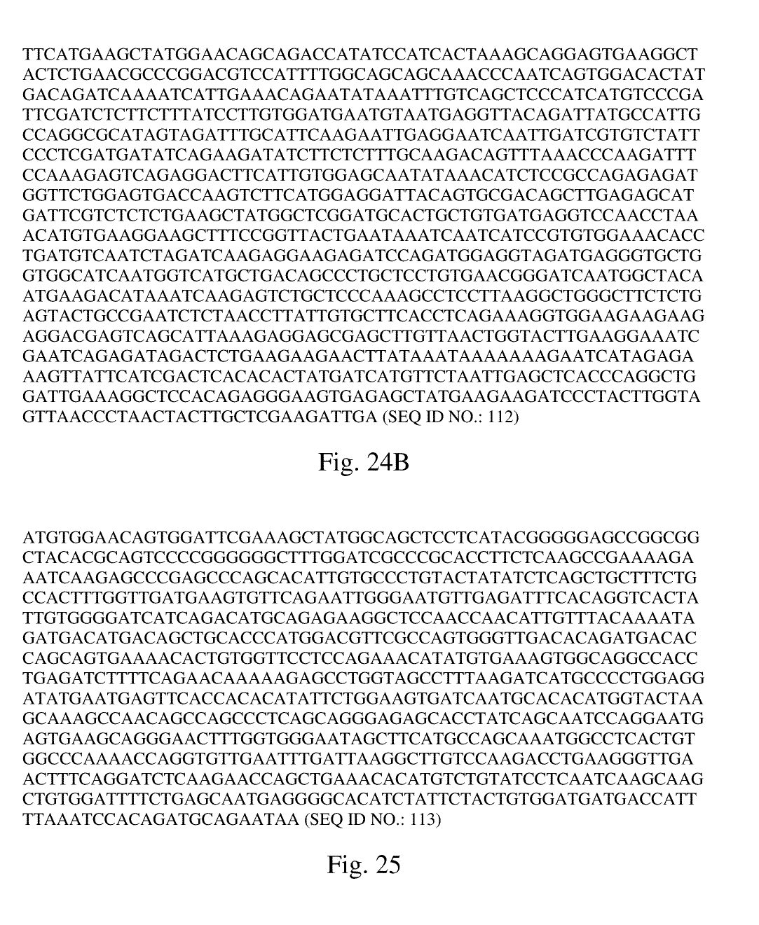

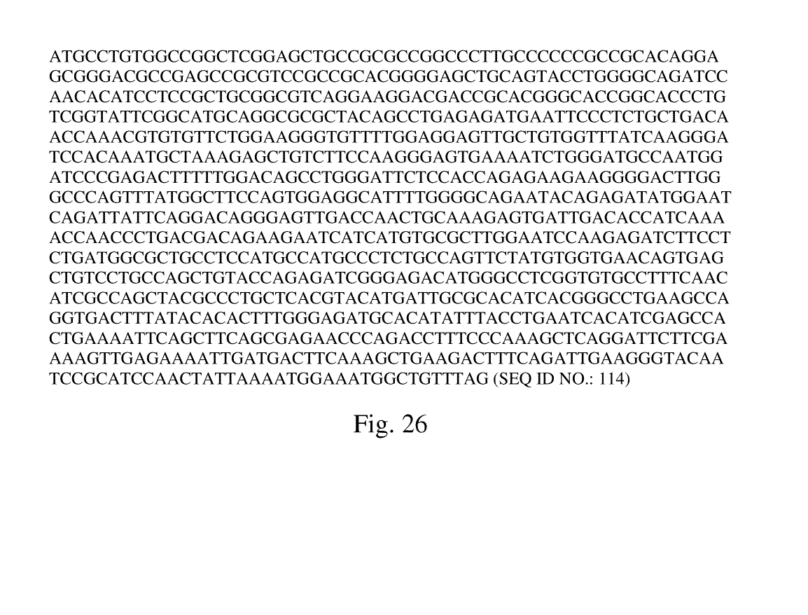

In other embodiments, the assay also detects a level of at least one proliferation biomarker. Exemplary proliferation biomarkers include PLK1, CCNA1 (nucleic acid sequence shown in FIG. 21), MCM4-6 (nucleic acid sequences shown in FIGS. 22-24 respectively), RPA2 (nucleic acid sequence shown in FIG. 25), and TYMS (nucleic acid sequence shown in FIG. 26). Methods of the invention may be conducted with any one of or a combination of the proliferative biomarkers in combination with the Suz12/Znf198 regulated biomarkers. In particular embodiments, the proliferative biomarkers are used in combination with the Suz12/Znf198 regulated biomarkers to grade the progression of a chronic hepatitis B virus infection toward a liver cancer. For example, overexpression of at least one of BAMBI, EpCAM, or PLK1 in combination with the expression levels of the Suz12/Znf198 regulated biomarkers indicates chronic hepatitis B virus infection progressing toward liver cancer. In certain embodiments, the overexpression of all three proliferative biomarkers in combination with the expression levels of the Suz12/Znf198 regulated biomarkers indicates chronic hepatitis B virus infection progressing toward liver cancer. In certain embodiments, the proliferative biomarker PLK1 is used in combination with the Suz12/Znf198 regulated biomarkers.

Methods of the invention may also determine aggressiveness of the liver cancer based upon which biomarker or combination of biomarkers are overexpressed. For example, overexpression of a combination of DKK1, DLK1, IGFII, and EpCAM indicates an aggressive form of liver cancer (Boyault et al., Hepatology, 45:42-52, 2007).

Methods of the invention involve conducting an assay that detects abnormal expression (over or under) of a biomarker regulated by Suz12/Znf198. Detailed descriptions of conventional methods, such as those employed to make and use nucleic acid arrays, amplification primers, hybridization probes, and the like can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

A differentially expressed gene or differential gene expression refer to a gene whose expression is activated to a higher or lower level in a subject suffering from a disorder, a chronic hepatitis B infection progressing toward liver cancer, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). The contents of each of theses references is incorporated by reference herein in their entirety. In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN (hydrolysis probes) PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN (hydrolysis probes) RT-PCR can be performed using commercially available equipment, such as, for example, ABI PRISM 7700 Sequence Detection System (real-time PCR detection instrument, Perkin-Elmer-Applied Biosystems, Foster City, Calif., USA), or LIGHTCYCLER (real-time PCR detection instrument, Roche Molecular Biochemicals, Mannheim, Germany). In certain embodiments, the 5′ nuclease procedure is run on a real-time quantitative PCR device such as the ABI PRISM 7700 Sequence Detection System (real-time PCR detection instrument). The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin. For performing analysis on pre-implantation embryos and oocytes, Chuk is a gene that is used for normalization.

A more recent variation of the RT-PCR technique is the real time quantitative PCR, which measures PCR product accumulation through a dual-labeled fluorigenic probe (i.e., TAQMAN probe (hydrolysis probe)). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996), the contents of which are incorporated by reference herein in their entirety.

In another embodiment, a MassARRAY-based gene expression profiling method is used to measure gene expression. In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BEADARRAY (bead based microarray assay) technology (Illumina, San Diego, Calif.; Oliphant et al., Discovery of Markers for Disease (Supplement to Biotechniques), June 2002; Ferguson et al., Analytical Chemistry 72:5618 (2000)); BEADARRAY (bead based microarray assay) for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety.

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell 88:243 51 (1997, the contents of each of which are incorporated by reference herein in their entirety).

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×106 microbeads/cm2). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated by reference herein in their entirety.

Methods of the invention may further involve providing a course of treatment to the patient based on results of the assessment of the liver of the patient. For example, the course of treatment may be continued monitoring and re-assessment at routine subsequent intervals, for example every two months, every four months, six months, every eight months, yearly, etc. Treatment may also be administration of anti-viral or chemotherapeutic drugs.

Aspects of the invention also recognize that PLK1 plays a critical role in the replication of the hepatitis B virus. Accordingly, administering a PLK1 inhibitor to a patient reduces or eliminates replication of the hepatitis B virus. Reducing or eliminating replication of the hepatitis B virus treats the chronic hepatitis B virus infection in the patient. Reducing or eliminating replication of the hepatitis B virus also prevents the chronic hepatitis B infection in the patient from progressing to a liver cancer.

Any PLK1 inhibitor known in the art may be used with such treatment methods. Exemplary PLK1 inhibitors are shown for example in U.S. patent application numbers 2011/0207716; 2011/0212942; and 2012/0115848, the content of each of which is incorporated by reference herein in its entirety. Other PLK1 inhibitor compounds are shown for example in PCT publication WO 2007/030361, the content of each of which is incorporated by reference herein in its entirety. In particular embodiment, the PLK1 inhibitor is any inhibitor shown in Steegmaier et al. (Curr Biol, 17:316-322, 2007), the content of which is incorporated by reference herein in its entirety.

The PLK1 inhibitors will often be used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

The PLK1 inhibitors herein may be used, for example, for the preparation of pharmaceutical compositions that comprise an effective amount of a PLK1 inhibitor, or a pharmaceutically acceptable salt thereof, as an active ingredient together or in admixture with a significant amount of one or more inorganic or organic, solid or liquid, pharmaceutically acceptable carriers.

The compositions herein are suitable for administration to a warm-blooded animal, including, for example, a human (or to cells or cell lines derived from a warm-blooded animal, including for example, a human cell), for the treatment or, in another aspect of the invention, prevention of (also referred to as prophylaxis against) a disease associated with the hepatitis B virus, comprising an amount of a compound of the present methods or a pharmaceutically acceptable salt thereof, which is effective for this inhibition, together with at least one pharmaceutically acceptable carrier.

The pharmaceutical compositions according to the methods are those for enteral, such as nasal, rectal or oral, or parenteral, such as intramuscular or intravenous, administration to warm-blooded animals (including, for example, a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The dose of a PLK1 inhibitor of the present methods or a pharmaceutically acceptable salt thereof to be administered to warm-blooded animals, for example humans of approximately 70 kg body weight, is for example, from approximately 3 mg to approximately 10 g, from approximately 10 mg to approximately 1.5 g, from about 100 mg to about 1000 mg/person/day, divided into 1-3 single doses which may, for example, be of the same size. Usually, children receive half of the adult dose.

The pharmaceutical compositions have from approximately, for example, 1% to approximately 95%, or from approximately 20% to approximately 90%, active ingredients. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes.

Solutions of the active ingredients, and also suspensions, and especially isotonic aqueous solutions or suspensions, are used, it being possible, for example in the case of lyophilized compositions that have the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The solutions or suspensions may have viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned, for example, liquid fatty acid esters that contain as the acid component a long-chained fatty acid having from 8-22, or from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, .beta.-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefosse, Paris), “Miglyol 812” (triglyceride of saturated fatty acids with a chain length of C₈ to C₁₂, Huls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredients with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

Suitable carriers are for example, fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragee cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredients in the form of granules, for example with fillers, such as lactose; binders, such as starches, and/or glidants, such as talc or magnesium stearate, and if desired with stabilizers. In soft capsules the active ingredients are preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragee coatings or the capsule casings, for example for identification purposes or to indicate different doses of active ingredient.

### EXAMPLES

The hepatitis B virus is a major risk factor for developing liver cancer, and the hepatitis B virus X protein (pX) has been implicated as a cofactor in liver cell transformation. The data herein show that hepatitis B virus replication as well as in vitro transformation by pX are associated with induction of Plk1 and down-regulation of the chromatin remodeling components Suz12 and Znf198. Herein, the same inverse relationship is demonstrated between Plk1 and Suz12/Znf198 in liver tumors from X/c-myc bitransgenic mice and woodchuck hepatitis virus-infected woodchucks. Employing these animal models of hepadnaviral pathogenesis and a cellular model of hepatitis B virus replication (HepAD38 cells) the effects of Suz12/Znf198 down-regulation on gene expression were examined. Genes analyzed included hepatic cancer stem cell markers BAMBI, DKK1,2, DLK1, EpCAM, MYC, and proliferation genes CCNA1, CCND2, IGFII, MCM4-6, PLK1, RPA2 and TYMS.

In untransformed hepatocytes, chromatin immunoprecipitation demonstrated Suz12 occupancy at the promoters of BAMBI, CCND2, DKK2, DLK1, EpCAM and IGFII. By contrast, Suz12 occupancy of these genes was reduced in pX-transformed and Suz12 knockdown cells (“Suz12 repressed”). The Suz12 repressed genes and proliferation genes were induced in hepatitis B virus-replicating HepAD38 cells, and they exhibited distinct expression profiles during hepatocellular carcinoma progression in X/c-myc bitransgenics. Specifically, CCND2, EpCAM and IGFII expression was elevated at the proliferative and preneoplastic stages in X/c20 myc bitransgenic livers, whereas BAMBI and PLK1 were overexpressed in hepatic tumors from X/c-myc bitransgenics and woodchuck hepatitis virus-infected woodchucks.

The distinct expression profile of the identified Suz12 repressed genes in combination with the proliferation genes demonstrate that those genes are biomarkers for progression of chronic hepatitis B virus infection progressing to hepatocellular carcinoma.

### Example 1

### Materials and procedures

Cell Culture:

Cell lines 4pX-1 (Tarn et al., J Biol Chem, 274:2327-2336, 1999), 4pX-1-SUZ12^{kd} (Suz12 knockdown), 4pX-1-ZNF198^{kd} (Znf198 knockdown) and 4pX-1^{GIPZ} (vector control cell line for 4pX-1-SUZ12^{kd} and 4pX-1-ZNF198^{kd} (Wang et al., Hepatology, 53:1137-47, 2011) are tetracycline-regulated pX-expressing cell lines, grown as described (Tarn et al., J Biol Chem, 274:2327-2336, 1999; and Wang et al., Hepatology, 53:1137-47, 2011). 4pX-1^{GIPZ} is equivalent to 4pX-1 cell line (Tarn et al., J Biol Chem, 274:2327-2336, 1999; and Wang et al., Hepatology, 53:1137-47, 2011). pX-transformed cultures were derived from 4pX-1 cells as described (Studach et al., Hepatology, 50: 414-423, 2009). HepAD38 cell line that supports hepatitis B virus replication by removal of tetracycline was grown as described (Ladner et al., Antimicrobial Agents and Chemotherapy, 41:1715-1720, 1997). Hepatitis B virus replication was quantified as described (Wang et al., Hepatology, 53:1137-47, 2011).

Real-Time PCR:

cDNA was synthesized from 2.0 μg total RNA using iSCRIPT cDNA synthesis kit (Bio-Rad). Real-time PCR reactions were performed in triplicates and normalized to GAPDH. Primer sequences are listed in Table 1.

TABLE 1

| Type | Gene | Forward Primer | Reverse Primer |
|---|---|---|---|
| Mouse | GAPDH | 5′ CAACAGCAACTCCCACTCT | 5′ATGTAGGCCATGAGGTCC |
| | | TCC 3′ (SEQ ID NO.: 1) | ACC 3′ (SEQ ID NO.: 2) |
| Mouse | BAMBI | 5′ GCCACTCCAGCTACTTCTT | 5′CACATGTAACCTGTTGCC |
| | | C 3′ (SEQ ID NO.: 3) | AC 3′ (SEQ ID NO.: 4) |
| Mouse | DKK1 | 5′ GAGGGGAAATTGAGGAAA | 5′ CCTTCTTGTCCTTTGGTGT |
| | | GC 3′ (SEQ ID NO.: 5) | G 3′ (SEQ ID NO.: 6) |
| Mouse | DKK2 | 5′ ACTCTTCCAAAGCCAGACT | 5′ GCATTCCAATCCAGGTTT |
| | | C 3′ (SEQ ID NO.: 7) | CC 3′ (SEQ ID NO.: 8) |
| Mouse | c-Myc | 5′ TGATCCTCAAAAAAGCCA | 5′ CGAAGCTGTTCGAGTTTG |
| | | CC 3′ (SEQ ID NO.: 9) | TG 3′ (SEQ ID NO.: 10) |
| Mouse | IGFII | 5′ ACACACACACACACACAC | 5′ TGGGATGGGCCTTGTTTT |
| | | AC 3′ (SEQ ID NO.: 11) | GG 3′ (SEQ ID NO.: 12) |
| Mouse | EpCAM | 5′ AGGGGCGATCCAGAACAA | 5′ ATGGTCGTAGGGGCTTTC |
| | | CG 3′ (SEQ ID NO.: 13) | TC 3′ (SEQ ID NO.: 14) |
| Mouse | RPA2 | 5′ TCAGTGGGTTGACACGGA | 5′ TGATCTTAAAGGCCACCA |
| | | TG 3′ (SEQ ID NO.: 15) | AGC 3′ (SEQ ID NO.: 16) |
| Mouse | CCNA1 | 5′ TCGCTACCTTCGAGAAGC | 5′ ATTCTTCCCCAACCTCCA |
| | | TG 3′ (SEQ ID NO.: 17) | CC 3′ (SEQ ID NO.: 18) |
| Mouse | CCND2 | 5′ AAGAAAGCACTCCCTGAC | 5′ TCCTCAAATGCCATCCCC |
| | | TG 3′ (SEQ ID NO.: 19) | TC 3′ (SEQ ID NO.: 20) |
| Mouse | DLK1 | 5′ GCTTCGCAAGAAGAAGAA | 3′ AGATCTCCTCATCACCAG |
| | | CC 3′ (SEQ ID NO.: 21) | CC 3′ (SEQ ID NO.: 22) |
| Woodchuck | GAPDH | 5′ TTTGGCAATGTGGAAGG | 5′ TGAGGGAAGATATTCTG |
| | | AC 3′ (SEQ ID NO.: 23) | GGC 3′ (SEQ ID NO.: 24) |
| Woodchuck | BAMBI | 5′ GTAGCAGAAACCTCATCA | 5′ AGGCCAACATAATCAGC |
| | | CC 3′ (SEQ ID NO.: 25) | AAC 3′ (SEQ ID NO.: 26) |
| Woodchuck | DKK1 | 5′ GTCAGCTCAATCCTAAGGA | 5′ ATTTACTGCAAACACAGG |
| | | TG 3′ (SEQ ID NO.: 27) | GG 3′ (SEQ ID NO.: 28) |
| Woodchuck | c-Myc | 5′ ACAACGAAAAGGCCCCCA | 5′ TAACTGTTCTCGCCGCTT |
| | | AG 3′ (SEQ ID NO.: 29) | CC 3′ (SEQ ID NO.: 30) |
| Woodchuck | EpCAM | 5′ CCGAAGAACTGACAAGG | 5′ GCAGTCTGCAAACTTTGA |
| | | ATAC 3′ (SEQ ID NO.: 31) | AC 3′ (SEQ ID NO.: 32) |
| Woodchuck | IGFII | 5′ GGGCAAGTTTTTCCAATAT | 5′ CCTCTCTGAACGCTTCA |
| | | GAC 3′ (SEQ ID NO.: 33) | AG 3′ (SEQ ID NO.: 34) |
| Woodchuck | MCM4 | 5′ TGTTTTCCAGTCCTCCTC | 5′ TACCCTAACCCCACTCCT |
| | | AG 3′ (SEQ ID NO.: 35) | TG 3′ (SEQ ID NO.: 36) |
| Woodchuck | MCM5 | 5′ TGCTGCCAACTCAGTGTT | 5′ TCTCCTCGTTGTGCTCAT |
| | | CG 3′ (SEQ ID NO.: 37) | CC 3′ (SEQ ID NO.: 38) |
| Woodchuck | MCM6 | 5′ GCAGTTCAAATACACACA | 5′ TGAGTCTCCTGAATACG |
| | | GCC 3′ (SEQ ID NO.: 39) | AACC 3′ (SEQ ID NO.: 40) |
| Woodchuck | TYMS | 5′ AATTTCCTCTGCTGACAA | 5′ GCATCCCAGATTTTCACT |
| | | CC 3′ (SEQ ID NO.: 41) | CC 3′ (SEQ ID NO.: 42) |
| Woodchuck | PLK1 | 5′ TGCAGCACTTACACAGTG | 5′ TTTGTCGGAGTAGTCCAC |
| | | TC 3′ (SEQ ID NO.: 43) | CC 3′ (SEQ ID NO.: 44) |
| Woodchuck | CCND2 | 5′ TCTTTCCCTCCTTCTCACC | 5′ AGGACCAACTTAGTCCC |
| | | AC 3′ (SEQ ID NO.: 45) | CAC 3′ (SEQ ID NO.: 46) |
| Woodchuck | RPA2 | 5′ AGGGATGTAAACCAGGAT | 5′ AGAAACTCCCAGGTGTTA |
| | | GG 3′ (SEQ ID NO.: 47) | GG 3′ (SEQ ID NO.: 48) |
| Human | GAPDH | 5′ GACCTGCCGTCTAGAAAA | 5′ TTGAAGTCAGAGGAGAC |
| | | AC 3′ (SEQ ID NO.: 49) | CAC 3′ (SEQ ID NO.: 50) |
| Human | BAMBI | 5′ GTATCAGCATGATGGTAG | 5′ AGCAACACTAAAATCAG |
| | | CAG 3′ (SEQ ID NO.: 51) | CCC 3′ (SEQ ID NO.: 52) |
| Human | DKK1 | 5′ TGAGTCCTTCTGAGATGA | 5′ TGAGAACCGAGTTCAAG |
| | | TGG 3′ (SEQ ID NO.: 53) | GTG 3′ (SEQ ID NO.: 54) |
| Human | DKK2 | 5′ CTGAAAGCATCTTAACCC | 5′ CATCTTAGTGTGTGGTCT |
| | | CTC 3′ (SEQ ID NO.: 55) | TCC 3′ (SEQ ID NO.: 56) |
| Human | c-Myc | 5′ GCCACGTCTCCACACATC | 5′ TCTTGGCAGCAGGATAG |
| | | AG 3′ (SEQ ID NO.: 57) | TCCTT 3′ (SEQ ID NO.: 58) |
| Human | EpCAM | 5′ CAGAAGAACAGACAAGG | 5′ TGCAGTCCGCAAACTTT |
| | | ACAC 3′ (SEQ ID NO.: 59) | TAC 3′ (SEQ ID NO.: 60) |
| Human | IGFII | 5′ TGGAGACGTACTGTGCT | 5′ CCAGGTGTCATATTGGAA |
| | | AC 3′ (SEQ ID NO.: 61) | GAA 3′ (SEQ ID NO.: 62) |
| Human | RPA2 | 5′ GCTTGTCCAAGACCTGAA | 5′ ACAGTAGAATAGATGTG |
| | | GG 3′ (SEQ ID NO.: 63) | CCCC 3′ (SEQ ID NO.: 64) |
| Human | CCNA1 | 5′ AAGAAAGCACTCCCTGA | 5′ TCCTCAAATGCCATCCC |
| | | CTG 3′ (SEQ ID NO.: 65) | CTC 3′ (SEQ ID NO.: 66) |
| Human | CCND2 | 5′ AAGTTTGCCATGTACCC | 5′ TTAGCCAGCAGCTCAG |
| | | ACC 3′ (SEQ ID NO.: 67) | TCAG 3′ (SEQ ID NO.: 68) |
| Human | PLK1 | 5′ AAGTCTCTGCTGCTCAA | 5′ ACGAACACGAAGTCGT |
| | | GCC 3′ (SEQ ID NO.: 69) | TGTC 3′ (SEQ ID NO.: 70) |
| Human | TYMS | 5′ GAATTCCCTCTGCTGAC | 5′ GCATCCCAGATTTTCAC |
| | | AAC 3′ (SEQ ID NO.: 71) | TCC 3′ (SEQ ID NO.: 72) |
| Human | PCNA | 5′ TCGATAAAGAGGAGGAA | 5′ CATACTGAGTGTCACCG |
| | | GCTG 3′ (SEQ ID NO.: 73) | TTG 3′ (SEQ ID NO.: 74) |
| Human | MCM4 | 5′ CCTCTCGTAAACGGAAAG | 5′ TGCTATGTCAGATTGTC |
| | | AAG 3′ (SEQ ID NO.: 75) | CCC 3′ (SEQ ID NO.: 76) |
| Human | MCM5 | 5′ TGCTGCCAACTCAGTGTT | 5′ TCTCCTCATTHTGCTCAT |
| | | C 3′ (SEQ ID NO.: 77) | CC 3′ (SEQ ID NO.: 78) |
| Human | MCM6 | 5′ ACCAGACACAAGATTCG | 5′ TCCAAGCACAGAAAAGT |
| | | AGAG 3′ (SEQ ID NO.: 79) | TCC 3′ (SEQ ID NO.: 80) |
| Human | DLK1 | 5′ TGTCCAACCTGCGCTACA | 5′ TGCTGAAGGTGGTCAT |
| | | AC 3′ (SEQ ID NO.: 81) | GTCG 3′ (SEQ ID NO.: 82) |
| ChIP | BAMBI | 5′ CCCTGCAAGCCCTAATA | 5′ CAATTAATCACCACCA |
| Assay | | AATG 3′ (SEQ ID NO.: 83) | CCACC 3′ (SEQ ID NO.: 84) |
| ChIP | CCND2 | 5′ TCTCCAACATCCACCCC | 5′ TCGCCAAACCAGGGATA |
| Assay | | TTC 3′ (SEQ ID NO.: 85) | AAG 3′ (SEQ ID NO.: 86) |
| ChIP | DKK2 | 5′ GTAGGAAAGGTGAGAGG | 5′ CCCCAATTCAAGAGTGAC |
| Assay | | CAG 3′ (SEQ ID NO.: 87) | AAAG 3′ (SEQ ID NO.: 88) |
| ChIP | EpCAM | 5′ ACCCAAAGCCCTCTTCTG | 5′ TGGCAGCTTACTTTCCAA |
| Assay | | AC 3′ (SEQ ID NO.: 89) | TACC 3′ (SEQ ID NO.: 90) |
| ChIP | IGFII | 5′ ACCCAACAGCAACAACA | 5′ AATGGGGGGCATCTTG |
| Assay | | AAC 3′ (SEQ ID NO.: 91) | ATCC 3′ (SEQ ID NO.: 92) |
| ChIP | c-Myc | 5′ TTCCCCAGCCTTAGAGA | 5′ GTTCTTGCCCTGCGTAT |
| Assay | | GAC 3′ (SEQ ID NO.: 93) | ATC 3′ (SEQ ID NO.: 94) |
| ChIP | DLK1 | 5′ AGGGGTAGTATAGGTAG | 5′ AAATCTCAAAAACCAA |
| Assay | | GAGGATT 3′ (SEQ ID NO.: 95) | ACCAAAC 3′ (SEQ ID NO.: |
| | | | 96) |
| ChIP | RPA2 | 5′ GTTGCTGGGAATTGAACT | 5′ TTCACACACACCCCATCT |
| Assay | | CG 3′ (SEQ ID NO.: 97) | CC 3′ (SEQ ID NO.: 98) |

Preparation of Whole Cell Extract (WCE) and Immunoblotting:

Mouse or woodchuck liver was homogenized in RIPA buffer (50 mM Tris pH7.4, 150 nM NaCl, 1% NP-40, 1 mM EDTA, 100 mM NaF, 50 mM glycerol phosphate, 1 mM sodium orthovanadate, 1 mM PMSF, 1 μg/ml aprotinin, 1 μg/ml leupeptin, 1 μg/ml pepstatin), sonicated on ice for 10 seconds and clarified by centrifugation (12,000 rpm, 15 minutes at 4° C.). Whole cell extract (10-20 μg) were analyzed by SDS-PAGE and immunoblotted using standard protocols. Antibodies: 1 Plk1, 1:1000 (Abcam), Suz12, 1:1000 (Abcam 12073), Znf198, 1:500 (Abcam), phospho-Plk1 T210, 1:500 (Abcam), phospho-Plk1 S137, 1:500 (Abcam), actin, 1:2000 (Sigma).

Chromatin Immunoprecipitation (ChIP) assays were performed as described (Wang et al., J Biol Chem, 283:25455-25467, 2008) employing 3.0 μg ChIP-validated Suz12 antibody (Abcam 12073). Immunoprecipitated DNA was quantified by real-time PCR. Primer sequences are listed in Table 1.

Statistical analyses were performed by Student's t-test (two-tailed).

### Example 2

### Elevated Plk1 and Reduced Protein Levels of Znf198 and Suz12 During Hepatocellular Carcinoma Pathogenesis in X/c-Myc Bitransgenic Mice

During oncogenic transformation of pX13 expressing hepatocytes (Studach et al., Hepatology, 50: 414-423, 2009), SUZ12 and ZNF198 exhibit a progressive decrease in protein but not mRNA levels, while Plk1 protein levels increase (Wang et al., Hepatology, 53:1137-47 15, 2011). Elevated Plk1 and reduced protein levels of Suz12 and Znf198 also occurs in cell lines derived from hepatitis B virus-induced liver tumors and in the HepAD38 cell line that supports hepatitis B virus replication (Wang et al., Hepatology, 53:1137-47, 2011). To establish the importance of these molecules in hepatitis B virus pX-mediated hepatocarcinogenesis, levels of Plk1, Znf198 and Suz12 were examined employing the X/c-myc bitransgenic mouse model that exhibits accelerated hepatocellular carcinoma development due to pX expression in the liver (Terradillos et al., Oncogene, 14:395-404, 1997).

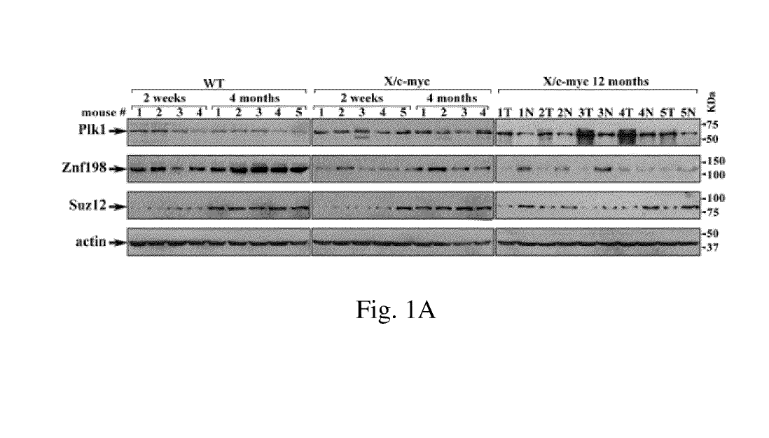

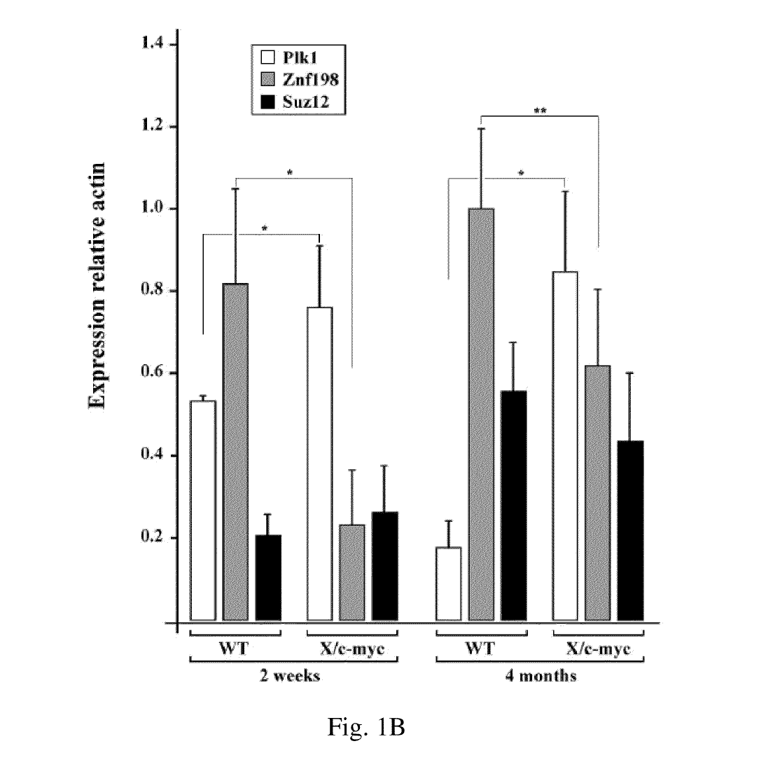

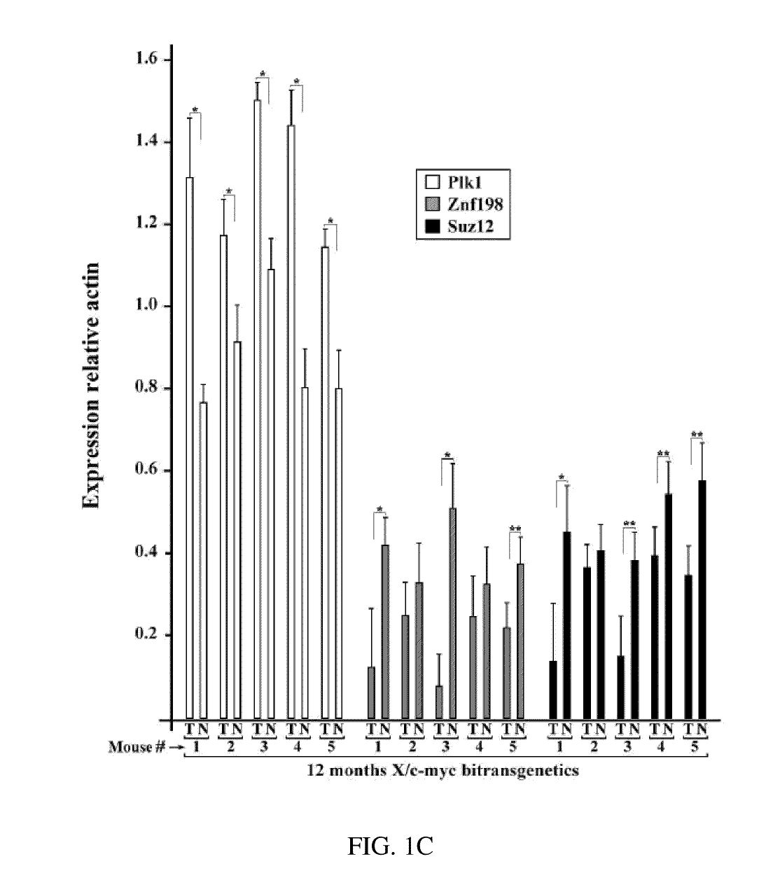

X/c-myc bitransgenics exhibit enhanced hepatocyte proliferation at 2 weeks, preneoplastic liver lesions at 4 months, and macroscopic liver tumors starting at 7-9 months (Terradillos et al., Oncogene, 14:395-404, 1997). Immunoblots of liver lysates from 2-week and 4-month X/c-myc bitransgenic mice exhibited elevated Plk1 protein compared to wild type mice (FIG. 1A). Likewise, increased Plk1 was observed in tumor (T) vs. peri-tumoral/normal (N) liver tissue at 12 months. On the other hand, protein levels of Znf198 were significantly reduced (p<0.01) as early as 2 weeks in X/c-myc bitransgenic livers, and remained reduced in T vs. N liver tissue. Suz12 protein levels were slightly reduced in the preneoplastic stage (4 months) of X/c-myc bitransgenic livers in comparison to wild type, but exhibited a significant reduction in most tumors at 12 months, compared to peri-tumoral tissue (FIG. 1A-C).

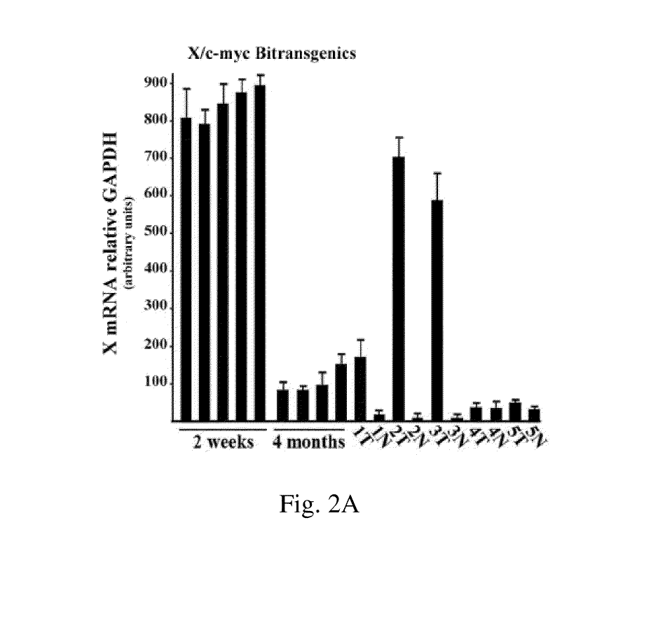

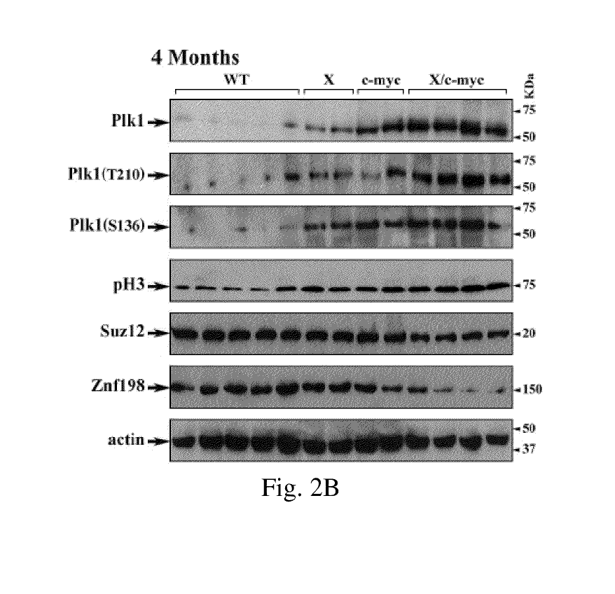

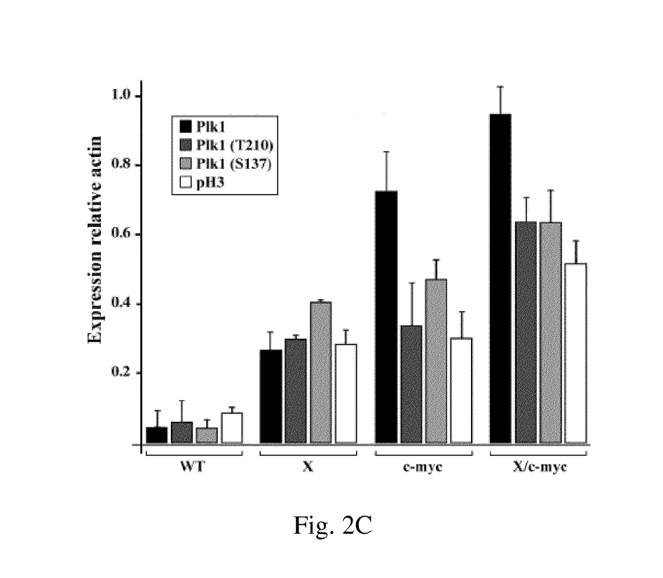

To understand the role of pX in accelerating hepatocellular carcinoa pathogenesis in X/c-myc bitransgenics, the level of X mRNA was determined by real time PCR. As shown previously (Terradillos et al., Oncogene, 14:395-404, 1997), highest expression of X was detected in X/c-myc bitransgenic liver at 2 weeks, decreasing to significantly lower levels by 4 months (FIG. 2A). Next, the level of Plk1 and activated Plk1 (phospho-T210 or phospho-S137 form) in the liver of 4-month wild type, X and c-myc monotransgenics, and X/c-myc bitransgenics was determined by immunoblots. Compared to wild type animals, levels of Plk1 and active Plk1 were increased in the liver of X and c-myc monotransgenics as well as X/c14myc bitransgenics. In agreement with the role of Plk1 in mitosis (Petronczki et al., Dev Cell, 14:646-659, 2008), levels of pH3 an early mitotic marker exhibited a concomitant increase, while protein levels of Suz12 and Znf198 exhibited a modest reduction in X and c-myc monotransgenics and a more pronounced reduction in X/c-myc bitransgenics (FIGS. 2B and C). These results are consistent with effects of both c-myc (D. Yu et al., Ann NY Acad Sci, 1059:145-159, 2005) and X (Lee et al., J Biol Chem, 277:8730-8740, 2002) in stimulating cell cycle progression and proliferation, and with the effect of pX in accelerating liver tumorigenesis of c-myc monotransgenic animals (Terradillos et al., Oncogene, 14:395-404, 1997).

### Example 3

### Enhanced Protein Level of Plk1 and Reduced Level of Suz12 in Liver Tumors of Chronically Woodchuck Hepatitis Virus-Infected Woodchucks

To assess the relevance of the observations derived from the X/cmyc mouse model, we performed similar analyses using liver tumors from woodchucks with established chronic woodchuck hepatitis virus infection. Experimental infection of neonate woodchucks with woodchuck hepatitis virus closely models woodchuck hepatitis virus-induced pathogenesis, including development of hepatocellular carcinoma (Menne et al., World J Gastroenterology, 13:104-124, 2007). We employed liver tumors from woodchuck hepatitis virus-infected woodchucks previously characterized and classified as well-differentiated (T₁), less-differentiated (T₂), and least-differentiated (T₃), based on anaplastic features and tumor size (Jacob et al., Hepatology, 39:1008-1016, 2004).

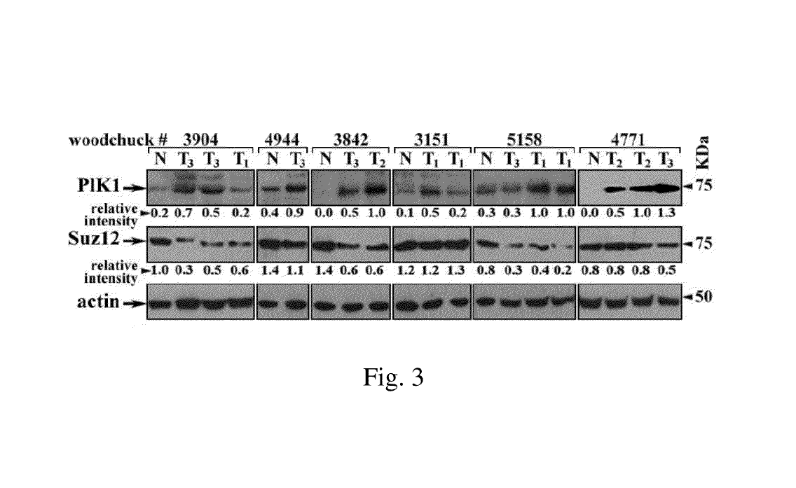

Protein levels of Plk1 and Suz12 were examined by immunoblots. Increased levels of Plk1 in tumor vs. peri-tumoral liver, and decreased levels of Suz12 in most tumors were observed (FIG. 3). Thus, opposite changes in Plk1 and Suz12 expression occur during hepatocarcinogenesis induced by chronic hepadnaviral infection, similar to hepatocellular carcinoma pathogenesis in X/c-myc bitransgenic mice (FIG. 1).

### Example 4

### Expression of Suz12/PRC2 Repressed Genes During pX-Mediated Transformation

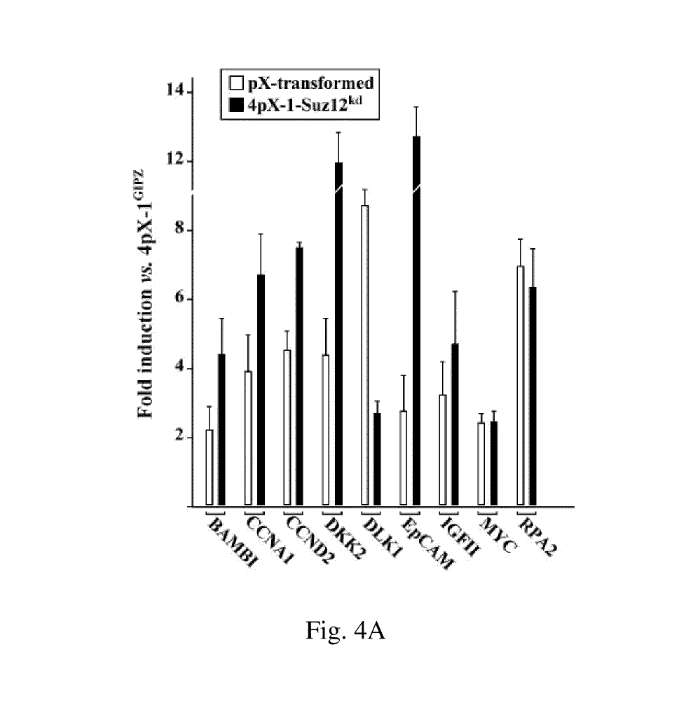

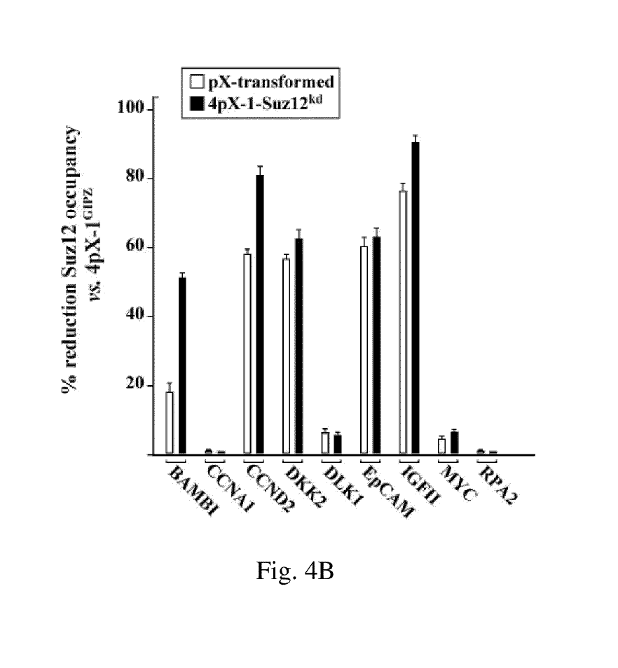

Since Suz12 is down-regulated in cellular (Studach et al., Hepatology, 50:414-423, 2009) and animal models (FIGS. 1 and 3) of hepadnaviral hepatocarcinogenesis, it was investigated as to whether genes known to be repressed by the Suz12/PRC2 complex become expressed during transformation. The PRC2 complex silences numerous genes in human embryonic fibroblasts including BAMBI, CCNA1, CCND2, DKK1, 2, DLK1, EpCAM, IGFII, MCM5, MYC and RPA2 (Bracken et al., Genes Dev, 209:1123-1136, 2006). Moreover, independent studies have confirmed that EpCAM and IGFII expression is regulated by dynamic changes in H3K27me3 (Lu et al., J Biol Chem, 285:8719-8732, 2010; and Li et al., Mol Cell Biol, 28:6473-82. 16, 2008). Expression of that subset of Suz12 repressed genes was investigated herein, because those genes are overexpressed in hepatic cancer stem cells (Yamashita et al., Gastroenterology, 136:1012-1024, 2009; Terris et al., J Hepatol, 52:280-291, 2010; and Yamashita et al., Cancer Res, 68:1451-1461, 2008) and in hepatocellular carcinomas of poor prognosis (Boyault et al., Hepatology, 45:42-52, 2007), as reviewed in Andrisani et al., (Seminars Cancer Biol, 21:4-9, 2011). Initially, real-time PCR was used to quantify mRNA levels in pX-transformed cells (Studach et al., Hepatology, 50: 414-423, 2009), Suz12 knockdown 4pX-1 cells (4pX-1-SUZ12^{kd}) used as positive control, and untransformed 4pX-1^{GIPZ} cells (Wang et al., Hepatology, 53:1137-47, 2011). Increased expression in pX-transformed cells and 4pX-1-SUZ12^{kd} cells, relative to 4pX-1^{GIPZ} cells (FIG. 4A and Table 2).

TABLE 2
Fold Induction Relative to 4pX-1 cells

| | cell line | | | | |
|---|---|---|---|---|---|
| gene | P2 | P3 | P4 | P5 | 4pX-1-Suz12^{kd} |
| BAMBI | 1.56 ± 0.45 | 2.18 ± 0.65 | 1.72 ± 0.49 | 2.30 ± 0.67 | 4.30 ± 0.87 |
| CCNA1 | 2.20 ± 0.57 | 2.36 ± 0.33 | 4.32 ± 1.17 | 3.96 ± 1.18 | 6.83 ± 1.02 |
| CCND2 | 2.46 ± 0.32 | 2.94 ± 0.29 | 4.28 ± 0.72 | 4.55 ± 0.51 | 7.65 ± 0.23 |
| DKK1 | 0.70 ± 0.65 | 0.13 ± 0.20 | 0.27 ± 0.20 | 2.03 ± 0.45 | 1.70 ± 0.45 |
| DKK2 | 3.05 ± 0.65 | 4.02 ± 1.03 | 3.08 ± 0.98 | 4.18 ± 1.55 | 12.3 ± 2.87 |
| DLK1 | 0.10 ± 0.12 | 4.48 ± 0.89 | 8.25 ± 1.23 | 10.1 ± 1.12 | 2.70 ± 0.43 |
| EpCAM | 2.00 ± 1.12 | 2.22 ± 0.56 | 2.28 ± 0.63 | 2.80 ± 1.01 | 12.7 ± 3.22 |
| IGFII | 2.22 ± 0.56 | 2.46 ± 1.01 | 3.51 ± 1.34 | 3.22 ± 0.87 | 4.70 ± 1.66 |
| MYC | 1.69 ± 0.40 | 1.48 ± 0.14 | 1.73 ± 0.71 | 2.48 ± 0.22 | 2.49 ± 0.37 |
| MCM5 | 3.23 ± 0.89 | 3.51 ± 0.71 | 2.82 ± 0.88 | 3.88 ± 0.20 | 2.12 ± 0.21 |
| PCNA | 2.22 ± 0.19 | 3.65 ± 0.40 | 3.52 ± 0.34 | 3.87 ± 0.76 | 2.42 ± 0.19 |
| RPA2 | 1.96 ± 0.44 | 4.24 ± 0.24 | 3.34 ± 0.35 | 7.07 ± 0.73 | 6.30 ± 1.15 |
| TYMS | 0.72 ± 0.34 | 0.51 ± 0.33 | 0.57 ± 0.13 | 3.96 ± 0.58 | 2.37 ± 1.55 |

Table 2 shows the expression of indicated genes during oncogenic transformation of X-expressing cells. P2-P5 cultures are derived by consecutive passages of partially polyploid pX-expressing cells, isolated by live cell-sorting from the immortalized 4pX-1 cell line, as described Studach et al. (Hepatology, 50: 414-423, 2009). P2 and P3 cultures are not transformed, whereas P4 and P5 cultures are pX-transformed cells (Studach et al., Hepatology, 50: 414-423, 2009). 4pX-1-Suz12^{kd} is a Suz12 knockdown cell line (Wang et al., Hepatology, 53:1137-47, 2011), used as positive control. Fold induction of indicated genes was quantified relative to expression in untransformed 4pX-1 cells. Results are from at least three independent RNA preparations used in real-time PCR reactions. Each PCR reaction was performed in triplicates and quantified relative to GAPDH used as internal control, +/−standard error of the mean.

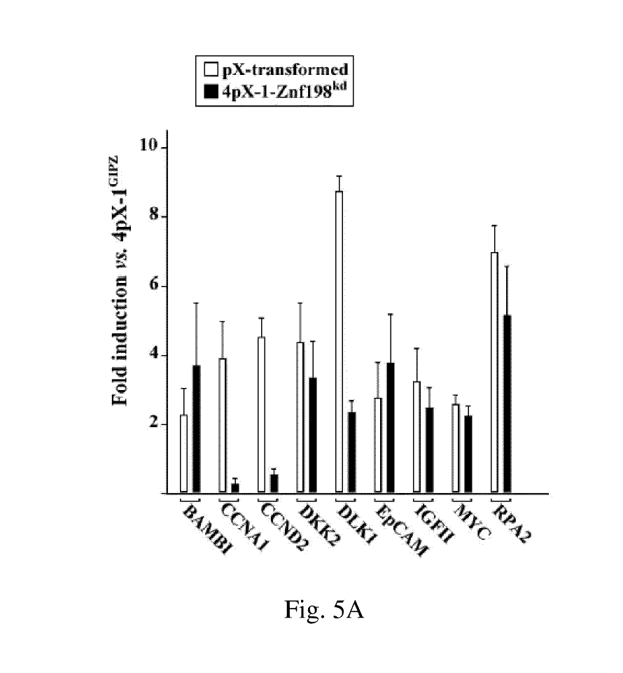

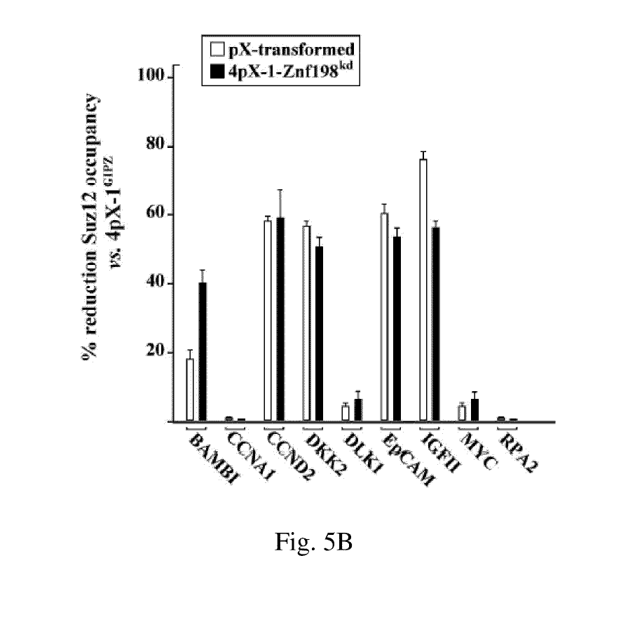

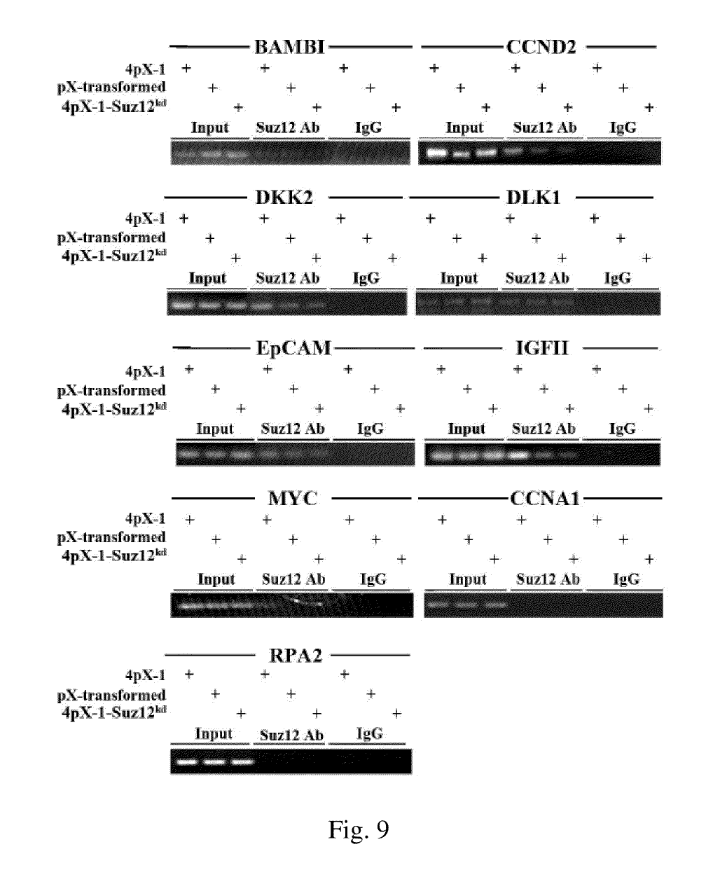

To determine whether these genes are directly repressed by the Suz12/PRC2 complex in untransformed 4pX-1^{GIPZ} cells, chromatin immunoprecipitation (ChIP) assays were employed with ChIP-validated Suz12 antibodies, as previously described (Wang et al., J Biol Chem, 283:25455-25467, 2008). Suz12 occupancy of their promoters was determined in untransformed 4pX-1^{GIPZ}, 4pX-1-SUZ12^{kd} and pX-transformed cells. Employing gene-specific primers, real-time PCR was used to determine the DNA sequences immunoprecipitated with Suz12 antibody. In comparison to 4pX-1GIPZ 8 cells, Suz12 occupancy was reduced in the promoters of CCND2, DKK2, EpCAM and IGFII in pX-transformed cells and 4pX-1-SUZ12^{kd} cells (FIG. 4B). A 20% reduction was quantified in Suz12 association with the BAMBI promoter in pX-transformed cells relative to 4pX-1^{GIPZ} cells, and a less than 10% reduction with the DLK1 promoter. By contrast, absence of Suz12 association with CCNA1, MYC and RPA2 promoters was independent of pX-mediated transformation, because this was also observed in untransformed 4pX-1 cells (FIG. 9). It was concluded that the genes BAMBI, CCND2, DKK2, DLK1, EpCAM and IGFII are transcriptionally repressed, to varying degrees, by the Suz12/PRC2 complex in untransformed 4pX-1^{GIPZ} cells. The Suz12/PRC2 complex is tethered via long intergenic RNA (lincRNA) to the LSD1-18 CoREST-HDAC1 complex stabilized by Znf198 (Tsai et al., Science, 329:689-693, 2010). Since protein levels of Znf198 are significantly reduced starting in the proliferative stage of X/c-myc bitransgenic liver (FIG. 1A), the effect of Znf198 knockdown on expression of the identified Suz12-repressed genes was examined. Employing the Znf198 knockdown 4pX-1 cell line (4pX-1-ZNF198KD21) described earlier (Wang et al., Hepatology, 53:1137-47, 2011), expression of the Suz12-repressed genes was quantified by real-time PCR (FIG. 5A) and Suz12 occupancy by ChIP assays (FIG. 5B). With the exception of CCNA1 and CCND2, the Suz12-repressed genes are induced in Znf198 knockdown cells (FIG. 5A). Conversely, with the exception of CCNA1, MYC and RPA2, Suz12 occupancy at these promoters was reduced in the context of Znf198 knockdown (FIG. 5B).

### Example 5

### Differential Expression of the Suz12 Repressed Genes During Hepatocellular Carcinoma Pathogenesis in X/c-Myc Bitransgenics

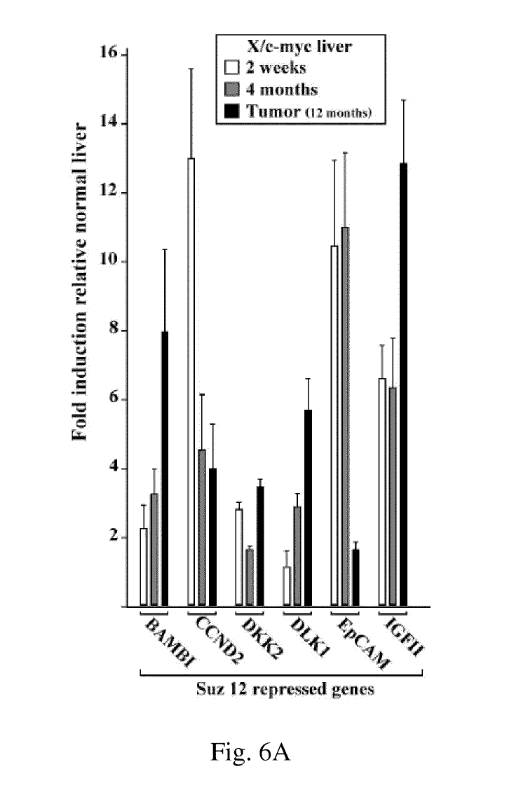

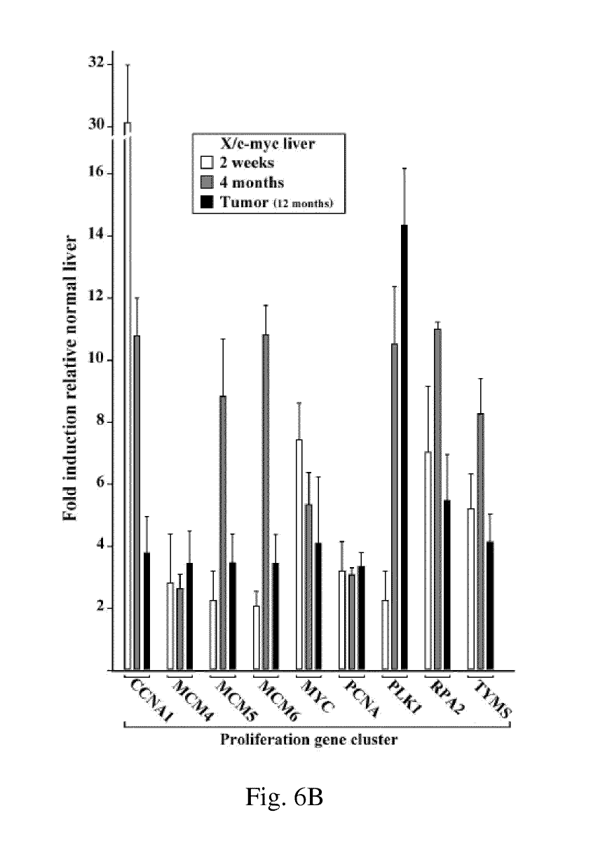

Next, the expression profile of the Suz12 target genes BAMBI, CCND2, DKK2, DLK1, EpCAM and IGFII and the proliferation genes CCNA1, MCM4-6, MYC, PCNA, PLK1, RPA2 and TYMS were investigated during liver tumorigenesis in X/c-myc bitransgenics (FIG. 6A and B, and Table 3).

TABLE 3

| | mouse | | |
|---|---|---|---|
| | 4 months | | |
| | Fold Induction Relative to WT | | |
| gene | X | c-Myc | X/c-Myc |
| BAMBI | 0.78 ± 0.65 | 0.80 ± 0.65 | 3.14 ± 1.07 |
| DKK1 | 0.30 ± 0.12 | 1.22 ± 0.34 | 2.24 ± 0.16 |
| DKK2 | 2.54 ± 0.78 | 2.80 ± 0.62 | 1.89 ± 0.58 |
| DLK1 | 2.32 ± 0.20 | 2.44 ± 0.30 | 2.68 ± 0.30 |
| EpCAM | 3.36 ± 1.24 | 2.01 ± 0.41 | 11.4 ± 2.98 |
| IGFII | 0.20 ± 0.16 | 0.40 ± 0.22 | 6.55 ± 1.76 |
| MYC | 1.37 ± 0.67 | 3.23 ± 0.41 | 4.24 ± 1.22 |
| CCNA1 | 2.45 ± 1.03 | 3.88 ± 1.14 | 10.9 ± 1.22 |
| CCND2 | 2.78 ± 1.48 | 3.14 ± 1.02 | 4.56 ± 1.54 |
| RPA2 | 1.98 ± 1.18 | 2.97 ± 0.67 | 11.5 ± 0.23 |
| MCM5 | 2.23 ± 0.55 | 2.43 ± 0.87 | 8.60 ± 2.03 |
| TYMS | 2.23 ± 0.55 | 3.19 ± 1.08 | 7.46 ± 1.09 |
| PCNA | 1.97 ± 0.35 | 2.08 ± 0.46 | 2.26 ± 0.35 |

Table 3 shows fold induction of indicated genes in the liver of 4-month old X monotransgenics, c-myc monotransgenics and X/c-myc bitransgenics in comparison to wild type mice of the same age, quantified by real-time PCR. Results are from at least three independent RNA preparations used in real-time PCR reactions. Each PCR reaction was performed in triplicates and quantification was relative to GAPDH used as internal control, +/−standard error of the mean.

CCND2, EpCAM and IGFII were highly up regulated in the liver of 2-week and 4-month X/c-myc bitransgenics relative to wild type liver. By contrast, at 12 months, fold induction of CCND2 and EpCAM was smaller when comparing tumor vs. peritumoral liver, while expression of BAMBI, DLK1 and IGFII progressively increased from the proliferative stage to the hepatocellular carcinoma stage at 12 months (FIG. 6A). Proliferation genes MCM5, MCM6, RPA2, TYMS and PLK1 exhibited enhanced expression at the preneoplastic stage (4 months) in comparison to wild type liver (FIG. 6B). Interestingly, expression of PLK1 significantly increased in hepatocellular carcinomas (FIG. 6B).

### Example 6

### Enhanced Expression of the Suz12-Repressed Genes During Hepatitis B Virus Replication

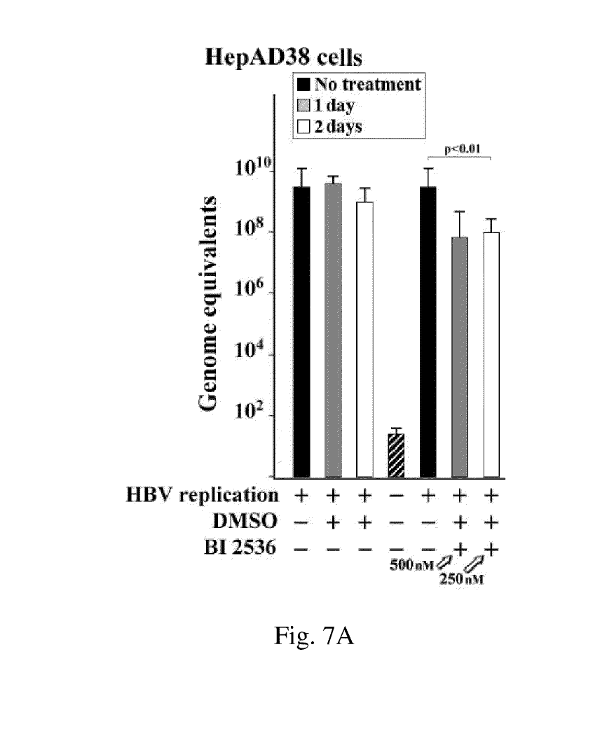

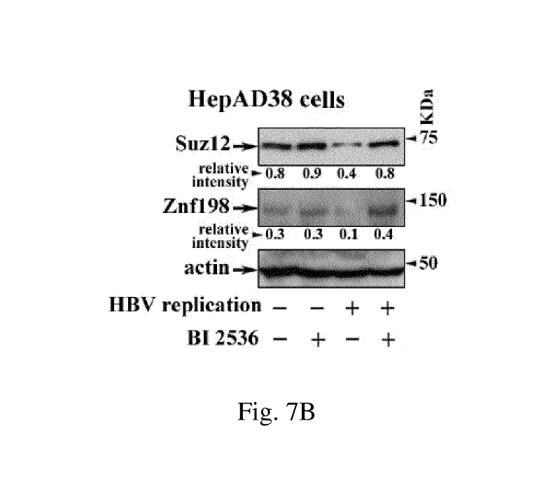

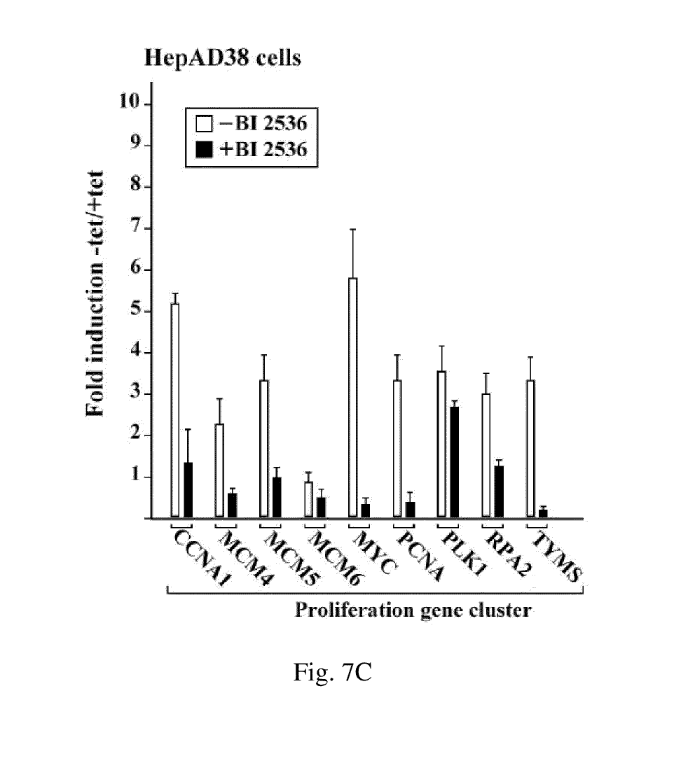

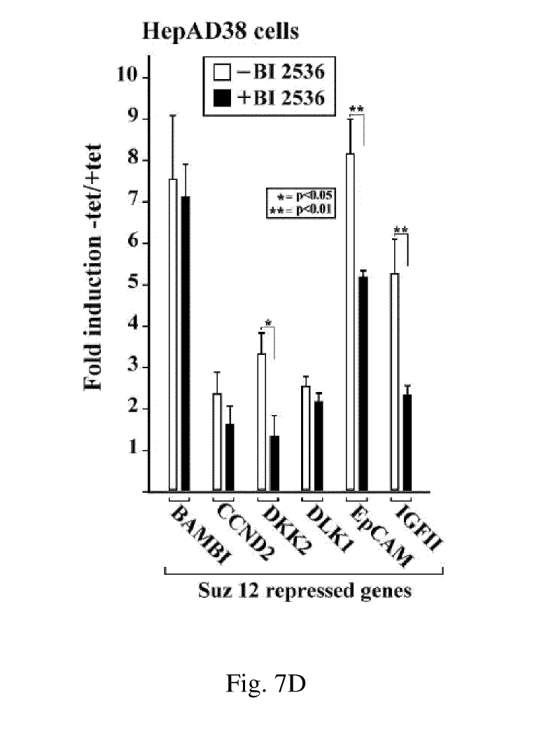

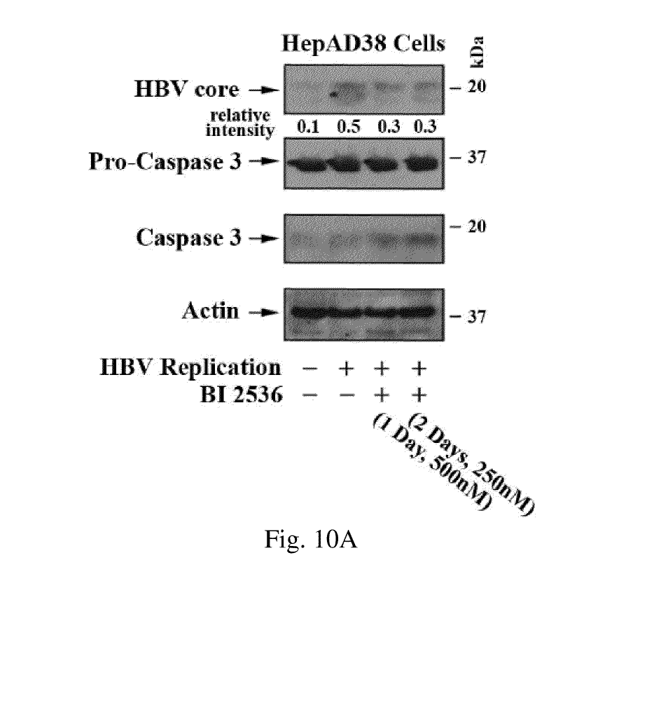

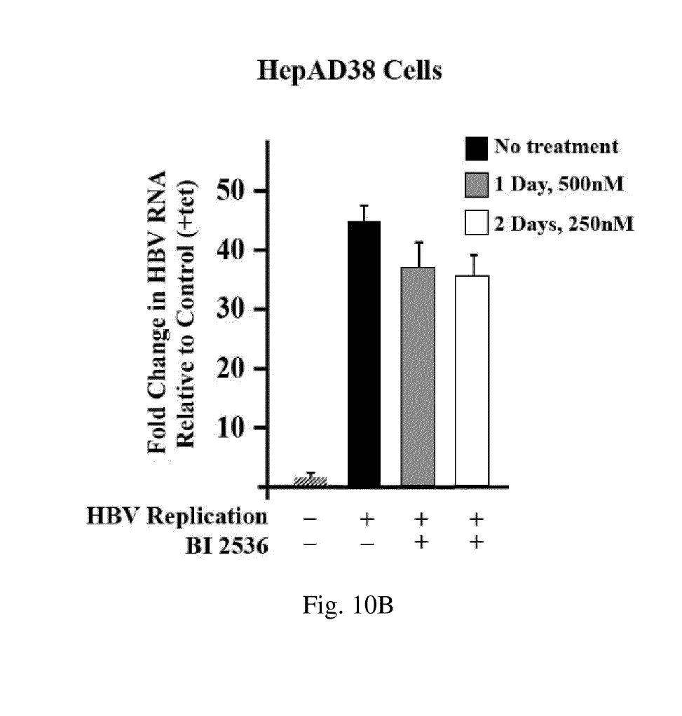

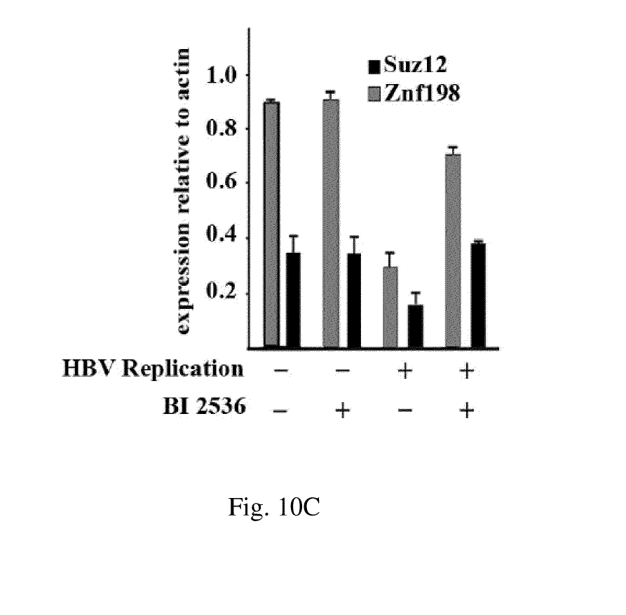

Protein levels of Suz12 and Znf198 are down-regulated in hepatitis B virus replicating cells, and siRNA knockdown of Suz12 and Znf198 increases hepatitis B virus replication (Wang et al., Hepatology, 53:1137-47, 2011). Accordingly, it was hypothesized that the Suz12 21 repressed genes might be up-regulated during hepatitis B virus replication. Employing the HepAD38 cell line that supports hepatitis B virus replication following tetracycline removal (Ladner et al., Antimicrobial Agents and Chemotherapy, 41:1715-1720, 1997), viral replication as well as expression of the Suz12-repressed and proliferation cluster genes were quantified 10 days after induction of hepatitis B virus replication (FIG. 7). Also, the effect of Plk1 inhibition on viral replication and expression of the proliferation cluster and Suz12 target genes was investigated. Treatment with the Plk1 inhibitor BI 2536 (Steegmaier et al., Curr Biol, 17:316-322, 2007) for 1 or 2 days, added on day 9 or day 8 of hepatitis B virus replication, respectively, suppressed hepatitis B virus replication, quantified by measuring hepatitis B virus genome equivalents in purified intracellular virions (FIG. 7A), immunoblots of hepatitis B virus core antigen (FIG. 10A), and PCR quantification of viral RNA (FIG. 10B). Under these conditions, inhibition of Plk1 did not induce apoptosis determined by the unchanged levels of pro-caspase-3 and caspase-3 (FIG. 10A), while protein levels of Znf198 and Suz12 were increased (FIG. 7B and FIG. 10C). Under these conditions of Plk1 inhibition, expression of the proliferation cluster and Suz12-repressed genes on day 10 of hepatitis B virus replication was quantified (FIG. 7C and D). Expression of both gene clusters increased in the presence of hepatitis B virus replication (FIG. 7C and D). Plk1 inhibition by BI 2536 addition significantly reduced expression of all the proliferation genes (FIG. 7C), demonstrating the link between Plk1 activity and cell cycle progression. Likewise, expression of the Suz12 repressed genes DKK2, EpCAM and IGFII was significantly decreased by BI 2536 treatment (FIG. 7D).

### Example 7

### Enhanced Expression of the SUZ12 Repressed Genes in Hepatic Tumors from Woodchuck Hepatitis Virus-Infected Woodchucks

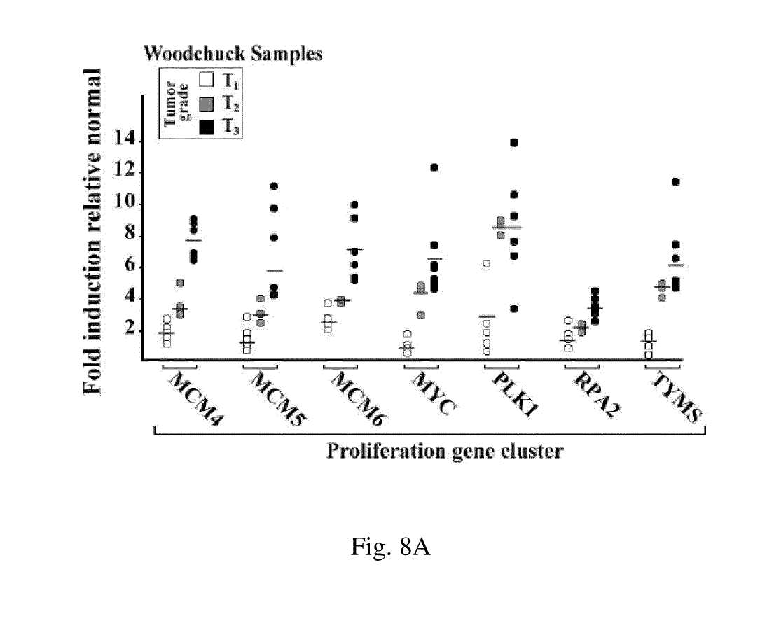

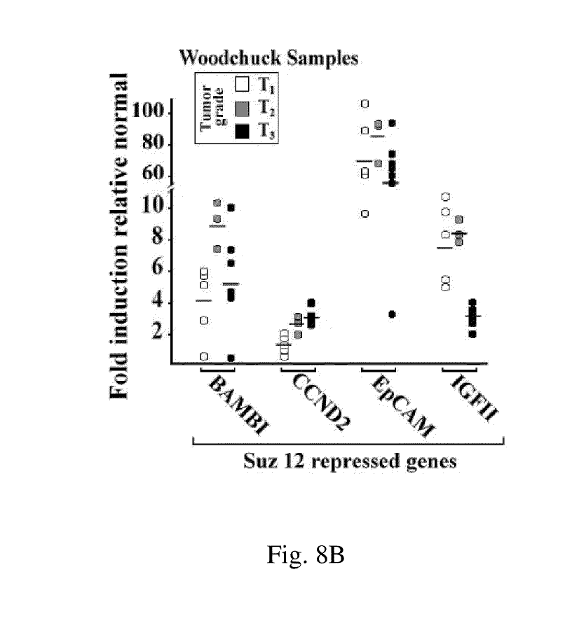

To determine the relevance of the observations derived from the 4pX-1 cell line 20 and the X/c-myc bitransgenics (FIGS. 1 and 4), the expression profile of the Suz12 repressed genes and the proliferation cluster genes in liver tumors from woodchucks chronically infected with woodchuck hepatitis virus were quantified. RNA from T₁, T₂, and T₃ liver tumors used in FIG. 3, were analyzed by real time PCR. Expression of both the proliferation cluster and the Suz12 repressed genes was induced in liver tumors from chronically woodchuck hepatitis virus-infected woodchucks in comparison to peritumoral tissue (FIG. 8 and Table 4).

Table 4 shows fold induction of the proliferation gene cluster and Suz12-repressed genes quantified in tumor vs peri-tumoral paired tissues from liver of individual woodchuck hepatitis virus-infected animals. The woodchuck identification number is from the study by Jacob et al. (Hepatology, 39:1008-1016, 2004). Results are from at least three independent RNA preparations used in real-time PCR reactions. Each PCR reaction was performed in triplicates and quantification was relative to GAPDH used as internal control, +/−standard error of the mean.

TABLE 4
Fold induction in Tumor (T) vs. peri-tumoral tissue

| | | Woodchuck number | | | | | | |
|---|---|---|---|---|---|---|---|---|
| | | 3904 | | | 4944 | | | 4151 |
| | Tumor Grade | T₁ | T3 | T3 | T₅ | T₂ | T3 | T₁ |
| Cluster | MCM4 | 3.24 ± 0.52 | 8.35 ± 0.66 | 8.89 ± 0.50 | 6.32 ± 0.64 | 4.54 ± 0.18 | 6.59 ± 0.01 | 1.30 ± 0.19 |
| | MCM5 | 2.49 ± 1.01 | 9.01 ± 0.50 | 7.86 ± 0.50 | 11.2 ± 0.83 | 4.36 ± 0.92 | 7.29 ± 1.19 | 0.35 ± 0.26 |
| | MCM6 | 2.61 ± 0.42 | 5.84 ± 0.26 | 8.63 ± 1.07 | 5.09 ± 0.78 | 3.76 ± 0.42 | 6.34 ± 0.95 | 1.96 ± 0.44 |
| | MYC | 0.52 ± 0.02 | 4.54 ± 1.70 | 12.3 ± 0.59 | 3.28 ± 0.32 | 0.39 ± 0.16 | 3.15 ± 0.03 | 1.18 ± 0.11 |
| | PLK1 | 6.12 ± 0.15 | 9.16 ± 1.77 | 7.22 ± 0.74 | 6.47 ± 1.03 | 8.88 ± 3.13 | 10.5 ± 2.35 | 2.27 ± 1.78 |
| | RPA2 | 1.13 ± 0.13 | 2.43 ± 0.54 | 3.79 ± 0.27 | 3.41 ± 1.39 | 2.12 ± 0.27 | 2.85 ± 0.17 | 1.87 ± 0.71 |
| | TYMS | 1.43 ± 0.67 | 4.25 ± 0.03 | 10.5 ± 2.14 | 6.88 ± 2.62 | 1.74 ± 1.03 | 4.34 ± 0.64 | 1.06 ± 0.88 |
| Genes | BAMBI | 3.11 ± 0.38 | 5.27 ± 0.40 | 4.48 ± 0.92 | 0.34 ± 0.16 | 10.3 ± 2.00 | 6.22 ± 0.23 | 5.39 ± 0.16 |
| | CCND2 | 0.97 ± 0.13 | 3.26 ± 0.48 | 3.24 ± 0.04 | 0.38 ± 0.23 | 2.57 ± 0.30 | 3.01 ± 0.11 | 1.77 ± 0.64 |
| | EPCAM | 10.0 ± 1.58 | 92.6 ± 3.62 | 67.8 ± 6.68 | 3.24 ± 0.52 | 67.8 ± 6.30 | 65.0 ± 2.82 | 89.7 ± 3.61 |
| | IGFII | 10.1 ± 1.66 | 3.91 ± 0.59 | 3.45 ± 0.69 | 3.91 ± 0.88 | 7.89 ± 0.99 | 2.98 ± 0.04 | 9.19 ± 1.12 |
| | | Woodchuck number | | | | | | |
| | | 4151 | 5158 | | | 4771 | | |
| | Tumor Grade | T₁ | T₁ | T₁ | T3 | T₂ | T₂ | T₃ |
| Cluster | MCM4 | 1.75 ± 0.33 | 1.79 ± 0.12 | 2.10 ± 0.04 | 7.10 ± 0.49 | 3.26 ± 0.20 | 3.49 ± 0.54 | 8.71 ± 1.03 |
| | MCM5 | 0.13 ± 0.04 | 1.37 ± 0.23 | 1.06 ± 0.29 | 4.77 ± 1.72 | 3.30 ± 0.91 | 2.51 ± 0.38 | 4.15 ± 0.06 |
| | MCM6 | 2.09 ± 0.28 | 3.38 ± 0.42 | 2.76 ± 0.59 | 8.81 ± 0.38 | 3.22 ± 0.77 | 3.95 ± 0.25 | 4.76 ± 0.30 |
| | MYC | 0.89 ± 0.44 | 1.79 ± 0.48 | 2.24 ± 0.42 | 5.36 ± 0.46 | 1.23 ± 0.76 | 2.20 ± 0.71 | 7.02 ± 0.96 |
| | PLK1 | 0.55 ± 0.36 | 0.91 ± 0.30 | 1.50 ± 0.23 | 3.23 ± 0.16 | 8.32 ± 1.08 | 7.60 ± 0.16 | 13.8 ± 0.90 |
| | RPA2 | 1.44 ± 0.54 | 1.39 ± 0.95 | 2.49 ± 0.11 | 2.75 ± 0.35 | 1.96 ± 0.19 | 2.08 ± 0.06 | 3.76 ± 0.13 |
| | TYMS | 0.80 ± 0.89 | 0.44 ± 0.37 | 0.32 ± 0.02 | 5.12 ± 1.43 | 4.00 ± 0.86 | 4.45 ± 1.62 | 5.81 ± 1.07 |
| Genes | BAMBI | 4.87 ± 0.24 | 0.44 ± 0.04 | 5.90 ± 0.03 | 4.14 ± 2.07 | 9.45 ± 2.51 | 7.31 ± 3.73 | 10.1 ± 2.11 |
| | CCND2 | 1.11 ± 0.46 | 1.92 ± 0.69 | 0.83 ± 0.08 | 3.66 ± 0.87 | 2.39 ± 0.07 | 2.09 ± 0.30 | 3.76 ± 0.79 |
| | EPCAM | 11.0 ± 35.0 | 68.9 ± 14.2 | 66.9 ± 1.18 | 31.8 ± 35.0 | 95.9 ± 8.62 | 95.1 ± 4.25 | 64.2 ± 4.29 |
| | IGFII | 8.11 ± 0.21 | 5.24 ± 0.33 | 5.48 ± 0.24 | 2.04 ± 0.80 | 8.07 ± 1.07 | 8.78 ± 3.25 | 3.12 ± 0.10 |

Specifically, expression of the proliferation cluster and in particular PLK1 was greatly induced in less-differentiated T₂ and T₃ tumors (FIG. 8A). EpCAM expression was significantly elevated in all hepatic tumors (FIG. 8B), and importantly, expression of BAMBI, similar to hepatocellular carcinomas from X/c-myc bitransgenics, was also induced in liver tumors from woodchuck hepatitis virus-infected animals.

### Example 8

### Biomarkers for Assessing a Liver of a Patient Having a Chronic Hepatitis B Virus Infection

The data herein show that an inverse relationship between Plk1 vs. Suz12 and Znf198 was observed in animal models of hepadnavirus-mediated hepatocarcinogenesis. These animal models include X/c-myc bitransgenic mice (Terradillos et al., Oncogene, 14:395-404, 1997) and woodchucks chronically infected with woodchuck hepatitis virus (Menne et al., World J Gastroenterology, 13: 104-124, 2007). In X/c-myc bitransgenics, expression of X in the liver cooperates with c-myc, reducing liver tumor latency by 2-3 months (Terradillos et al., Oncogene, 14:395-404, 1997). Since X monotransgenics do not develop liver tumors, this animal model has established the weak oncogenic potential of the hepatitis B virus X protein. On the other hand, chronic woodchuck hepatitis virus infection in woodchucks recapitulates hepatitis B virus infection and hepatitis B virus-mediated hepatocarcinogenesis in humans (Menne et al., World J Gastroenterology, 13: 104-124, 2007; Gouillat et al., J Hepatol, 26:1324-1330, 1997; and Tennant et al., Gastroenterology; 127:S283-S293, 2004). Woodchuck hepatitis virus is similar in structure and viral life cycle to hepatitis B virus, causing acute or chronic hepatitis in infected woodchucks (Gouillat et al., J Hepatol, 26:1324-1330, 1997; Tennant et al., Gastroenterology; 127:S283-S293, 2004; and Cote et al., Hepatology, 31:190-200, 2000).

Increased Plk1 is observed in both animal models of liver tumorigenesis (FIGS. 1 and 3). Also, increased Plk1 expression and activation is detected in the liver of X and c-myc 23 monotransgenics (FIG. 2), in agreement with the role of c-myc and pX in enhancing proliferation. The lower Plk1 activation in X vs. c-myc monotransgenics is congruent with the weak oncogenic potential of pX (Terradillos et al., Oncogene, 14:395-404, 1997). In X/c-myc bitransgenics, expression and activation of Plk1 is further increased, as well as down-regulation of Suz12 and Znf198, demonstrating a cooperative effect between c-myc and pX. This cooperative effect is via Plk1-mediated direct down regulation of Suz12 and Znf198 or induction of microRNAs targeting Suz12 and Znf198. For example, miR-200 shown to target Suz12 (Iliopoulos et al., Molec Cell, 39:761-772, 2010) is significantly up-regulated in human cirrhotic liver.

Znf198 protein levels were significantly reduced in the proliferative stage of X/c-myc bitransgenics at 2 weeks (FIG. 1). On the other hand, Suz12 protein levels were slightly reduced in the preneoplastic stage at 4 months, and significantly reduced in most liver tumors in comparison to peri-tumoral tissue, in both animal models (FIGS. 1 and 3). The variation in Suz12 protein levels in tumor vs. peri-tumoral tissue suggests that multiple mechanisms contribute to inactivation of the PRC2 complex. For example, EZH2, an essential component of PRC2, is inactivated by Akt-mediated phosphorylation (Svotelis et al., EMBO J, 30:3947-3961, 2011). Significantly, Akt activation is a hallmark of poor prognosis hepatitis B virus-mediated hepatocellular carcinomas (Boyault et al., Hepatology, 45:42-52, 2007). Other mechanisms that inactivate PRC2 include loss of JARID required for association of PRC2 with target genes (Pasini et al., Nature, 464: 306-310, 2007), or induction of the H3K27me3 demethylase, as in Epstein Bar virus-infected cells and Hodgkin's lymphoma (Anderton et al., Oncogene, 30:2037-2043, 2011). Furthermore, combined effects of the loss of the Suz12/PRC2 and Znf198/LSD1-CoREST21-HDAC1 complexes may contribute to oncogenic transformation. This notion is supported by studies showing that these complexes are linked via the lincRNA HOTAIR, thereby coupling repressive H3K27me3 mediated by PRC2 with demethylation of transcriptionally activating H3K4me3 (Tsai et al., Science, 329:689-693, 2007). Indeed, Suz12 occupancy is reduced in the promoters of select Suz12-repressed genes in the context of Znf198 knockdown, thereby allowing their expression (FIG. 5).

Although Suz12/PRC2 silences more than 1,000 genes in human embryonic fibroblasts (Bracken et al., Genes Dev, 209:1123-1136, 2007), the data herein show that a subset of Suz12/PRC2 repressed genes are relevant to hepatocellular carcinoma-mediated liver cancer. One group of Suz12/PRC2 repressed genes include proliferation genes over-expressed in human hepatocellular carcinoma, and the other group include genes that are over-expressed in hepatic cancer stem cells.

ChIP assays with Suz12 antibody demonstrated that BAMBI, CCND2, DKK2, 11 DLK1, EpCAM and IGFII were direct targets of transcriptional repression by Suz12/PRC2 in untransformed hepatocytes (FIG. 4B). The data show that these genes are induced during pX-mediated transformation in vitro (FIG. 4A). The expression profile of these Suz12-repressed genes and proliferation cluster of genes is distinct in the liver of X/c-myc 16 bitransgenics during progression to hepatocellular carcinoma (FIG. 6). Specifically, CCND2, EpCAM and IGFII exhibit enhanced expression in the liver of 2-week X/c-myc bitransgenics (proliferative stage), whereas at the preneoplastic stage (4 months) the proliferation markers CCNA1, MCM6, RPA2, TYMS and PLK1 exhibit the highest expression. At the hepatocellular carcinoma stage, expression of the Suz12-repressed genes BAMBI, DLK1 and IGFII and the proliferation marker PLK1 are significantly elevated. Liver tumors from woodchuck hepatitis virus-infected woodchucks also exhibit the distinct expression of BAMBI, high expression PLK1 and also high expression of EpCAM (FIG. 8). The absence of EpCAM induction in liver tumors of X/c-myc bitransgenics is due to their well differentiated status and lack of metastatic potential.

The data also show enhanced expression of both proliferation cluster and Suz12 target genes in the HepAD38 cell line that supports hepatitis B virus replication (FIG. 7). Importantly, inhibition of Plk1 by treatment with BI2536 significantly reduced viral replication as well as expression of both the proliferation cluster and select Suz12 target genes (FIG. 7C and D), showing that Plk1 inhibitors have antiviral and anticancer effects.

Thus, the distinct expression profile of the genes repressed by Suz12/PRC2 in untransformed hepatocytes, in combination with the proliferation marker PLK1, distinguishes the proliferative and preneoplastic stages from the hepatocellular carcinoma stage in both animal models of hepatocellular carcinoma pathogenesis. CCND2, EpCAM and IGFII are highly expressed in the proliferative liver-stage of X/c-myc bitransgenics and hepatitis B virus-replicating HepAD38 cells, demonstrating that these genes are prognostic biomarkers of chronic hepatitis B virus infection progressing to hepatocellular carcinoma. On the other hand, BAMBI in combination with high expression of EpCAM and PLK1 are biomarkers for the hepatocellular carcinoma stage.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for assessing a liver of a patient having a chronic hepatitis B virus infection, the method comprising:
obtaining a sample from a patient having a chronic hepatitis B virus infection;
conducting an assay on the sample to determine a level of at least one biomarker regulated by Suz12/Znf198; and
assessing a liver of the patient based on the level of the at least one biomarker.

2. The method according to claim 1, wherein prior to the obtaining step, the method further comprises determining that the patient has a chronic hepatitis B virus infection.

3. The method according to claim 1, further comprising providing a course of treatment to the patient based on results of the assessing step.

4. The method according to claim 1, wherein the biomarker is selected from the group consisting of BAMBI, CCND2, DKK2, DLK1, EpCAM, IGFII, and a combination thereof.

5. The method according to claim 4, wherein the assay also detects a level of at least one proliferation biomarker.

6. The method according to claim 5, wherein the proliferation biomarker is selected from the group consisting of PLK1, CCNA1, MCM4-6, RPA2, TYMS

7. The method according to claim 6, wherein assessing comprises determining whether the patient having a chronic hepatitis B virus infection will develop a liver cancer.

8. The method according to claim 7, wherein overexpression of at least one of CCND2, EpCAM, or IGFII indicates chronic hepatitis B virus infection progressing toward liver cancer.

9. The method according to claim 7, wherein overexpression of at least one of BAMBI, EpCAM, and PLK1 indicates presence of liver cancer.

10. The method according to claim 7, further comprising determining aggressiveness of the liver cancer.

11. The method according to claim 10, wherein overexpression of a combination of DKK1, DLK1, IGFII, and EpCAM indicates an aggressive form of liver cancer.

12. A method for reducing or eliminating replication of a hepatitis B virus, the method comprising: administering a PLK1 inhibitor to thereby reduce or eliminate replication of a hepatitis B virus.

13. The method according to claim 12, wherein the hepatitis B virus is present in a patient suffering from a chronic hepatitis B virus infection.

14. The method according to claim 13, wherein reducing or eliminating replication of a hepatitis B virus treats the chronic hepatitis B virus infection in the patient.

15. The method according to claim 12, wherein the PLK1 inhibitor is formulated with a pharmaceutically acceptable carrier.

16. The method according to claim 12, wherein the PLK1 inhibitor is provided as a unitary dose.

17. A method for preventing a chronic hepatitis B infection in a patient from progressing to a liver cancer, the method comprising:
determining that a patient has a chronic hepatitis B infection that is progressing toward a liver cancer; and
administering a PLK1 inhibitor to thereby reduce or eliminate replication of a hepatitis B virus, thereby preventing the chronic hepatitis B infection in the patient from progressing to a liver cancer.

18. The method according to claim 17, wherein determining comprises conducting an assay on a sample from the patient to determine a level of at least one biomarker regulated by Suz12/Znf198, wherein overexpression of at least one of CCND2, EpCAM, or IGFII indicates that the chronic hepatitis B virus infection is progressing toward liver cancer.

19. The method according to claim 17, wherein the PLK1 inhibitor is formulated with a pharmaceutically acceptable carrier.

20. The method according to claim 17, wherein the PLK1 inhibitor is provided as a unitary dose.
